(19) United States
(12) Patent Application Publication
(10) Pub. No.: US 2023/0346986 A1
(43) Pub. Date: Nov. 2, 2023

(54) METHODS AND AGENTS FOR THE DETECTION AND TREATMENT OF CANCER

(71) Applicant: CASE WESTERN RESERVE UNIVERSITY, Cleveland, OH (US)

(72) Inventors: James BASILION, Cleveland, OH (US); Andrew JOHNSON, Cleveland, OH (US); Dong LUO, Cleveland, OH (US); Thomas MEADE, Cleveland, OH (US)

(21) Appl. No.: 17/625,989

(22) PCT Filed: Jul. 10, 2020
(86) PCT No.: PCT/US2020/041625

§ 371 Date: Jan. 10, 2022

Related U.S. Application Data
(60) Provisional application No. 62/872,478, filed on Jul. 10, 2019.

(51) Int. Cl. A61K 49/18; A61K 47/69; B82Y 5/00; A61K 47/62; A61K 41/00; A61P 35/00

(52) U.S. Cl. CPC A61K 49/1881; A61K 47/6929; B82Y 5/00; A61K 47/62; A61K 41/0038; A61P 35/00

(57) ABSTRACT

A nanoparticle agent includes a gold nanoparticle, at least one thiol modified Gd(III) macrocycle complex, and at least one prostate specific membrane antigen (PSMA) ligand, wherein the PSMA ligand and the thiol modified Gd(III) complex are each individually coupled to the gold nanoparticle via one or more thiol (SH) groups.

### RELATED APPLICATION

This application claims priority from U.S. Provisional Application No. 62/872,478, filed Jul. 10, 2019, the subject matter of which is incorporated herein by reference in its entirety.

### GOVERNMENT FUNDING

This invention was made with government support under Grant No. R01EB020353 awarded by The National Institutes of Health. The United States government has certain rights in the invention.

### TECHNICAL FIELD

This application relates to prostate-specific membrane antigen (PSMA) ligand targeted gold-gadolinium (Gd(III)-Au) nanoparticles and their use in compositions for targeting, imaging, and treating cancer.

### BACKGROUND

Cancer detection and treatment are hindered by the inability to differentiate between cancer cells and normal cells. Better detection tools for cancer or tumor imaging are needed for earlier diagnosis of cancers. Molecular recognition of tumor cells would facilitate early detection, guided surgical resection, evaluation of response to therapy as well as targeted drug delivery including radiotherapeutics and nanoparticles. In order to improve surgical resection, targeted imaging tools must specifically label tumor cells, not only in the main tumor but also along the edge of the tumor and in the small tumor cell clusters that disperse throughout the body.

Targeted imaging tools designed to label molecules that accumulate in the tumor microenvironment may also be advantageous as therapeutic targeting agents, as they can identify both the main tumor cell population and areas with infiltrating cells that contribute to tumor recurrence. The ability to directly target therapeutics to the tumor cell and/or its microenvironment would increase both the specificity and sensitivity of current treatments, therefore reducing non-specific side effects of chemotherapeutics that affect cells throughout the body. Prostate-specific membrane antigen (PSMA) is a 120 kDa protein expressed in prostate tissues and was originally identified by reactivity with a monoclonal antibody designated 7E11-05 (Horoszewicz et al., 1987, Anticancer Res. 7:927-935; U.S. Pat. No. 5,162,504). PSMA is characterized as a type II transmembrane protein sharing sequence identity with the transferrin receptor (Israeli et al., 1994, Cancer Res. 54:1807-1811). PSMA is a glutamate carboxy-peptidase that cleaves terminal carboxy glutamates from both the neuronal dipeptide N-acetylaspartylglutamate (NAAG) and gamma-linked folate polyglutamate. That is, expression of PSMA cDNA confers the activity of N-acetylated α-linked acidic dipeptidase or “NAALADase” activity (Carter et al., 1996, PNAS 93:749-753).

PSMA is expressed in increased amounts in prostate cancer, and elevated levels of PSMA are also detectable in the sera of these patients (Horoszewicz et al., 1987, supra; Rochon et al., 1994, Prostate 25:219-223; Murphy et al., 1995, Prostate 26:164-168; and Murphy et al., 1995, Anticancer Res. 15:1473-1479). As a prostate carcinoma marker, PSMA is believed to serve as a target for imaging and cytotoxic treatment modalities for prostate cancer. Prostate carcinogenesis, for example, is associated with an elevation in PSMA abundance and enzymatic activity of PSMA. PSMA antibodies, particularly indium-111 labeled and tritium labeled PSMA antibodies, have been described and examined clinically for the diagnosis and treatment of prostate cancer. PSMA is expressed in prostatic ductal epithelium and is present in seminal plasma, prostatic fluid and urine.

Recent evidence suggests that PSMA is also expressed in tumor associated neovasculature of a wide spectrum of malignant neoplasms including conventional (clear cell) renal carcinoma, transitional cell carcinoma of the urinary bladder, testicular embryonal carcinoma, colonic adenocarcinoma, neuroendocrine carcinoma, gliobastoma multiforme, malignant melanoma, pancreatic ductal carcinoma, non-small cell lung carcinoma, soft tissue carcinoma, breast carcinoma, and prostatic adenocarcinoma. (Chang et al. (1999) Cancer Res. 59, 3192-3198).

Gold has excellent radiation enhancing capability. In the development of gold nanoparticle-based radiosensitizers, high tumor targeting and fast body clearance is the key, as an unnecessary over-exposure of radiation to healthy tissue and potential gold particle-induced toxicity are not desirable. There remains a need for an ideal radiosensitizer developed for cancer, such a prostate cancer, the leading cancer diagnosed in men.

### SUMMARY

Embodiments described herein relate to agents and methods for use in detecting, monitoring, and/or imaging cancer cells and/or cancer cell metastasis, migration, dispersal, and/or invasion, and/or treating cancer in a subject in need thereof. The agent includes a gold nanoparticle, at least one thiol modified gadolinium 3⁺ (Gd(III)) macrocycle complex, and at least one prostate specific membrane antigen (PSMA) ligand coupled to the gold nanoparticle for targeting the composition to a PSMA expressing cancer cell. The PSMA ligand and the Gd(III) complex are coupled to the gold nanoparticle via one or more thiol (SH) groups. In some embodiments, the gold nanoparticle is less than about 6 nm in core diameter.

The at least one thiol modified Gd(III) macrocycle complex can include a lipoic acid modified and amine functionalized Gd(III) macrocycle complex. In some embodiments, the at least one thiol modified Gd(III) macrocycle complex can include an amine functionalized and 1,2 dithiolane modified Gd(III) macrocycle complex. In some embodiments, the at least one thiol modified Gd(III) macrocycle complex can have the formula:

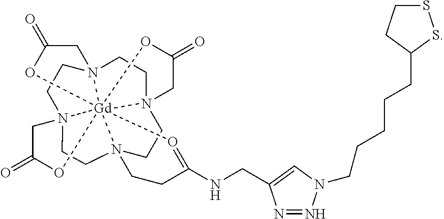

The at least one PSMA ligand coupled to the gold nanoparticle for targeting the composition to a PSMA expressing cancer cell can include have the general formula (I):

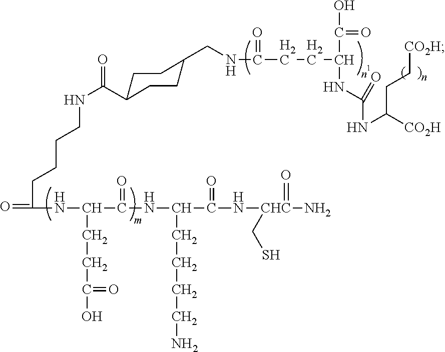

wherein m, n and n¹ are each independently 1, 2, 3, or 4.

In some embodiments, the PSMA ligand can have the formula (II):

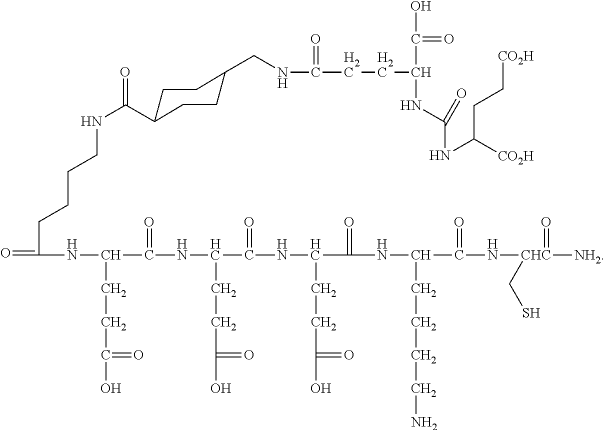

The cancer detected, imaged or treated with the agent can include a PSMA expressing cancer. The PSMA cancer can include but is not limited to glioma, lung cancer, melanoma, breast cancer, and prostate cancer.

The agent when used as a molecular probe can be detected in vivo by detecting, recognizing, or imaging the agent by magnetic resonance imaging (MRI), positron emission tomography (PET) imaging, computer tomography (CT) imaging, gamma imaging, near infrared imaging, or fluorescent imaging.

Other embodiments described herein also relate to methods of detecting, monitoring, and/or imaging cancer cells and/or cancer cell metastasis, migration, dispersal, and/or invasion in a subject. The method includes administering a diagnostically effective amount of an agent to the subject. The agent includes a gold nanoparticle, at least one thiol modified gadolinium 3⁺ (Gd(III)) macrocycle complex, and at least one prostate specific membrane antigen (PSMA) ligand coupled to the gold nanoparticle for targeting the composition to a PSMA expressing cancer cell. The PSMA ligand and the Gd(III) complex are coupled to the gold nanoparticle via one or more thiol (SH) groups. In some embodiments, the gold nanoparticle is less than about 6 nm in core diameter.

The method further includes detecting the nanoparticle agents selectively targeted to the cancer cells to determine the location and/or distribution of the cancer cells in the subject.

The at least one thiol modified Gd(III) macrocycle complex can include a lipoic acid modified and amine functionalized Gd(III) macrocycle complex. In some embodiments, the at least one thiol modified Gd(III) macrocycle complex can include an amine functionalized and 1,2 dithiolane modified Gd(III) macrocycle complex. In some embodiments, the at least one Gd(III) or complex can have the formula:

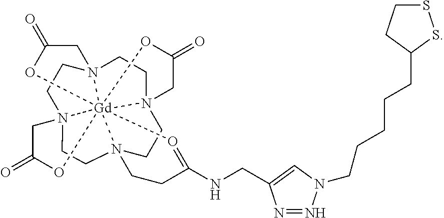

The at least one PSMA ligand coupled to the gold nanoparticle for targeting the composition to a PSMA expressing cancer cell can include have the general formula (I):

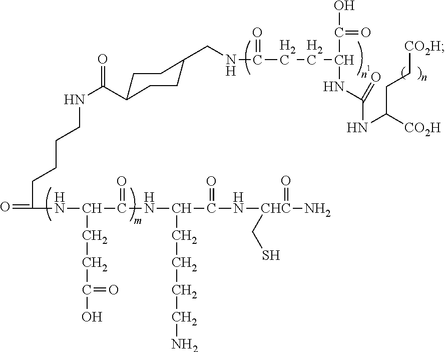

wherein m, n and n¹ are each independently 1, 2, 3, or 4.

In some embodiments, the PSMA ligand can have the formula (II):

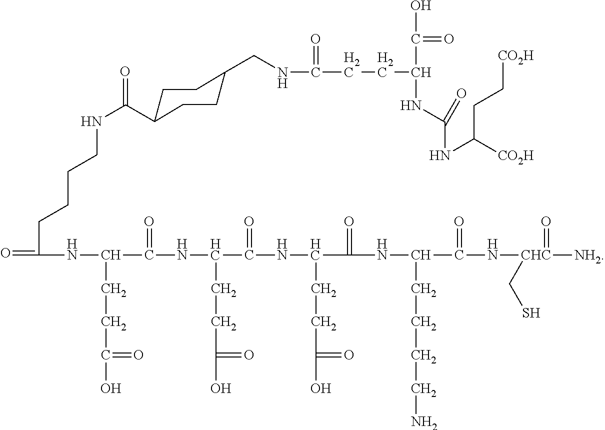

The nanoparticle agent can be detected in vivo by detecting, recognizing, or imaging the agent by at least one of magnetic resonance imaging (MRI), positron emission tomography (PET) imaging, computer tomography (CT) imaging, gamma imaging, near infrared imaging, or fluorescent imaging. In certain embodiments, the nanoparticle agent can be detected in vivo by detecting, recognizing, or imaging the agent by MRI.

Still other embodiments relate to a method of treating cancer in a subject in need thereof. The method can include administering to the subject a therapeutically effective amount of an agent that includes a gold nanoparticle, at least one thiol modified Gd(III) macrocycle complex, and at least one prostate specific membrane antigen (PSMA) ligand coupled to the gold nanoparticle for targeting the composition to a PSMA expressing cancer cell. The PSMA ligand and the Gd(III) complex are coupled to the gold nanoparticle via one or more thiol (SH) groups.

The method further includes detecting the nanoparticle agents selectively targeted to the cancer cells to determine the location and/or distribution of the cancer cells in the subject. In some embodiments, the targeted nanoparticle agent can be detected in vivo by detecting, recognizing, or imaging the agent by at least one of MRI, positron emission tomography (PET) imaging, computer tomography (CT) imaging, gamma imaging, near infrared imaging, or fluorescent imaging.

The method also includes irradiating the detected cancer, thereby inducing the radiosensitizing effects of the gold nanoparticle. In some embodiments, the gold nanoparticle is less than about 6 nm in core diameter. The thiol modified Gd(III) macrocycle complex coupled to the gold nanoparticle surface can provide a synergistic effect on the radiosensitization of cancer cells.

The at least one thiol modified Gd(III) macrocycle complex can include a lipoic acid modified and amine functionalized Gd(III) macrocycle complex. In some embodiments, the at least one thiol modified Gd(III) macrocycle complex can include an amine functionalized and 1,2 dithiolane modified Gd(III) macrocycle complex. In some embodiments, the at least one thiol modified Gd(III) macrocycle complex can have the formula:

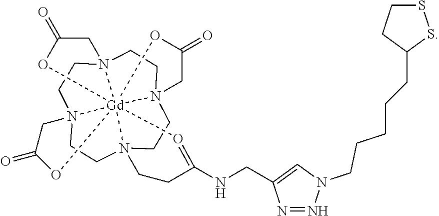

The at least one PSMA ligand coupled to the gold nanoparticle for targeting the composition to a PSMA expressing cancer cell can include have the general formula (I):

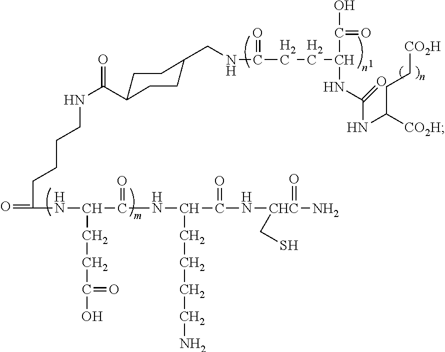

wherein m, n and n¹ are each independently 1, 2, 3, or 4.

In some embodiments, the PSMA ligand can have the formula (II):

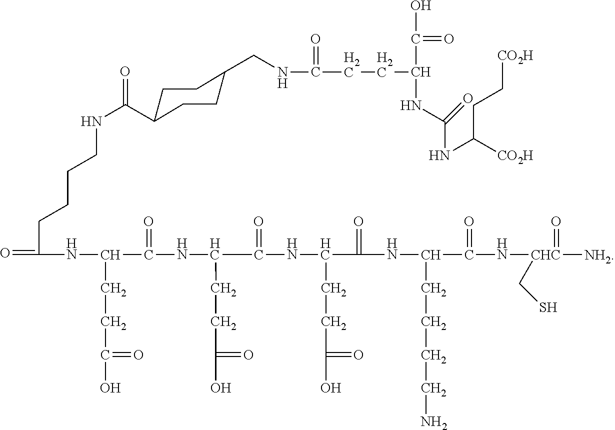

In some embodiments, the agent is administered systemically, such as by intravenous injection. The cancer can be a PSMA expressing cancer selected from glioma, lung cancer, melanoma, breast cancer, or prostate cancer. The presence of the nanoparticle agent can be detected in the subject by at least one of MRI, positron emission tomography (PET) imaging, computer tomography (CT) imaging, gamma imaging, near infrared imaging, or fluorescent imaging. In some embodiments, the cancer is irradiated with gamma ray irradiation.

### BRIEF DESCRIPTION OF THE DRAWINGS

FIGS. 1A-D illustrate Au—Gd(III)-PSMA NPs for MR-guided radiation therapy. (A) Schematic representation of Au—Gd(III)-PSMA NPs with AuNPs as the core, Gd(III) complex as stabilizer on the surface, and Cys-PSMA-1 as targeting ligand. Yttrium (Y) complex with chelated Gd(III) replaced by Y(III) was also synthesized and conjugated to AuNP surfaces as control. (B) TEM image of Au— Gd(III)-PSMA NPs with average core size of 5 nm. (C) DLS shows the hydrodynamic diameter of Au—Gd(III)-PSMA NPs. (D) Agarose gel electrophoresis demonstrates the successful binding of Cys-PSMA-1 to AuNPs and stability of Au—Gd(III)-PSMA NPs in serum.

FIGS. 2(A-D) illustrate in vitro cell targeting, binding affinity and MR contrast. (A) Selective uptake of Au— Gd(III)-PSMA NPs by PC3pip and PC3flu cells. Cells were incubated with NPs for 24 h and then silver stained for visualization. Quantitative Au and Gd(III) content in PC3pip and PC3flu cells was measured with ICP-MS. (B) Competition binding curves for parent ZJ24 ligands, Cys-PSMA-1 ligands, and Au—Gd(III)-PSMA NPs (n=3). (C) Image of PC3pip and PC3flu cell pellets shows NP uptake by PC3pip cells (pink color) and the corresponding T1-weighted MR images of the cell pellets acquired at 7 T. PC3pip cells incubated with Au—Gd(III)-PSMA NPs demonstrate the highest contrast enhancement. (D) Increased signal-to-noise ratio for PC3pip and PC3flu cells after incubating with Au—Gd(III)-PSMA NPs. Data are presented as mean±SD (n=3), and differences between groups are compared with two-tailed t-tests, **p<0.01.

FIGS. 3(A-F) illustrates in vitro radiation enhancement by Au—Gd(III)-PSMA NPs and selective cell killing. (A) Cytotoxicity of Au—Gd(III)-PSMA NPs and Au—Y(III)-PSMA NPs after incubation for 24 h. (B, C) Survival curves of PC3pip and PC3flu cells with and without addition of Au—Gd(III)-PSMA NPs (B) and Au—Y(III)-PSMA NPs (C) at radiation doses of 0, 2, 4, 6 and 8 Gy. (D) Schematic demonstration of selective cell killing experiment with mixed PC3pip and PC3flu colonies. (E) Plates showing mixed colonies stained with silver. PC3pip colonies stained black and PC3flu colonies were relatively transparent. Representative images are shown of n=3. (F) Quantification of the ratio of PC3pip colony number to PC3flu colony number. Data are presented as mean±SD (n=3), and differences between groups are compared with two-tailed t-tests, **p≤0.01.

FIGS. 4(A-D) illustrates in vivo tumor targeting of Au—Gd(III)-PSMA NPs and MR imaging. T1-weighted spin echo images of mice with PC3pip tumor (A) and PC3flu tumor (B) obtained at 7 T. Tumors are indicated by red triangles and bladders are indicated by green arrows. Representative images are shown of n=3. (C) Contrast-to-noise ratio (CNR) of PC3pip and PC3flu tumors relative to muscle, computed from T1-weighted T1-weighted images. (D) Au and Gd(III) content in PC3pip and PC3flu tumors 24 h after Au—Gd(III)-PSMA NPs injection. Data are presented as mean±SD (n=3), and differences between groups are compared with two-tailed t-tests, **p<0.01.

FIGS. 5(A-F) illustrates in vivo Au—Gd(III)-PSMA NP-enhanced radiotherapy. (A) Timeline of Au—Gd(III)-PSMA NPs injection, radiation treatments and diffusion-weighted imaging (DWI)scanning time points. (B) Tumor growth curves without radiation (PBS) and with one irradiation (6 Gy) after receiving PBS (PBS-X), Au—Gd(III)-PSMA NPs or Au—Y(III)-PSMA NPs. (C) Tumor growth curves for mice injected with PBS or Au—Gd(III)-PSMA NPs after receiving irradiation (6 Gy) twice. Data are presented as mean±SD (n=5). (D) Body weight of mice after each treatment. (E) ADC maps of mice injected with PBS or Au—Gd(III)-PSMA NPs before and at 2 h, 4 h, and 24 h after a single irradiation (6 Gy). Representative images are shown of n=3. (F) Increased ADC values at 2 h, 4 h, and 24 h after radiation (6 Gy) for mice injected with PBS or Au—Gd(III)-PSMA NPs. Data are presented as mean±SD (n=3), and differences between groups are compared with two-tailed t-tests, *p≤0.05, **p≤0.01.

FIG. 6 illustrates convergent synthetic scheme for Dithioline-Gd(III) complex. The Y(III) complex was prepared in a similar fashion.

FIG. 7 illustrates ESI mass spectrum of Cys-PSMA-1 ligand at 1190 (m/z).

FIGS. 8(A-B) illustrates UV-Vis absorbance spectroscopy was employed to ascertain the stability of Au—Gd-PSMA NPs in 10% FBS solution by monitoring the surface plasmon resonance (SPR) band of gold (˜520 nm). The data suggests that the nanoparticles are stable over 28 days with (A) no shift of absorbance band and (B) the absorbance intensity does not change significantly compared to that at day 0.

FIG. 9 illustrates an example of r1 relaxivity calculation for free Gd(III) complex.

FIG. 10 illustrates r1 relaxivity calculation for free Au—Gd NPs.

FIG. 11 illustrates r1 relaxivity calculation for free Au—Gd-PSMA NPs.

FIG. 12 illustrates MRI Solution phantom of Au—Gd-PSMA NPs at different Gd(III) concentrations.

FIG. 13 illustrates Au and Gd(III) content in collected urine samples at 8 h and 24 h post-injection of Au— Gd-PSMA NPs as determined by ICP-MS. Data are presented as mean±SD (n=3).

FIG. 14 illustrates Au and Gd(III) content in main organs at 24 h post-injection of Au—Gd-PSMA NPs as determined by ICP-MS. Data are presented as mean±SD (n=3).

FIG. 15 illustrates ADC maps of mice before and after injection of Au—Gd-PSMA NPs. NP injection does not affect the apparent diffusion coefficient of H₂O.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

The term “agent” is used herein to denote a chemical compound, a mixture of chemical compounds, a biological macromolecule, or an extract made from biological materials.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “nanoparticle” refers to any particle having a diameter of less than 1000 nanometers (nm). In some embodiments, nanoparticles can be optically or magnetically detectable. In some embodiments, intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments. In general, the nanoparticles should have dimensions small enough to allow their uptake by eukaryotic cells. Typically, the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., about 1 nm to about 30 nm or about 1 nm to about 5 nm, are used in some embodiments.

“PSMA” refers to Prostate Specific Membrane Antigen, a potential carcinoma marker that has been hypothesized to serve as a target for imaging and cytotoxic treatment modalities for cancer.

As used herein, the terms “treating” or “treatment” of a disease can refer to executing a treatment protocol to eradicate at least one diseased cell. Thus, “treating” or “treatment” does not require complete eradication of diseased cells.

The phrases “parenteral administration” and “administered parenterally” are art-recognized terms and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

As used herein, an “effective amount” can refer to that amount of a therapeutic agent that results in amelioration of symptoms or a prolongation of survival in the subject and relieves, to some extent, one or more symptoms of the disease or returns to normal (either partially or completely) one or more physiological or biochemical parameters associated with or causative of the disease.

The term “radiosensitizer” refers to compounds or agents that increase the cytotoxicity of ionizing radiation. For example, heavy-metal nanomaterials with high atomic number (Z) values, such as gold nanoparticles.

The terms “patient”, “subject”, “mammalian host,” and the like are used interchangeably herein, and refer to mammals, including human and veterinary subjects.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to targeted gold nanoparticle agents for use in detecting, monitoring, and/or imaging cancer cells and/or cancer cell metastasis, migration, dispersal, and/or invasion in a subject, methods of detecting, monitoring, and/or imaging cancer cells and/or cancer cell metastasis, migration, dispersal, and/or invasion in a subject, methods of determining and/or monitoring the efficacy of a cancer therapeutic and/or cancer therapy administered to a subject in need thereof, and methods of treating a cancer in a subject in need thereof.

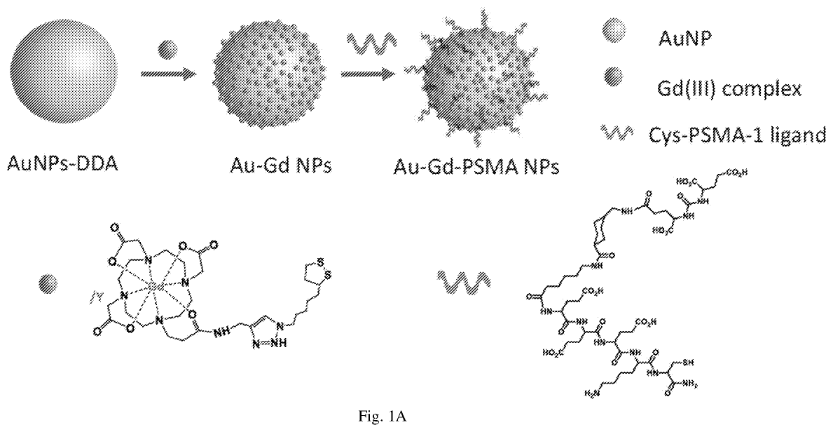

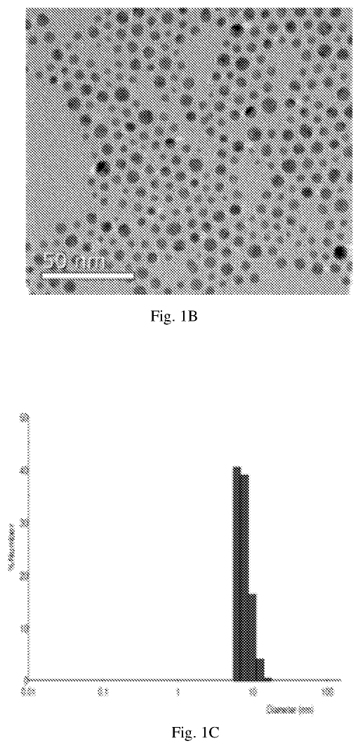

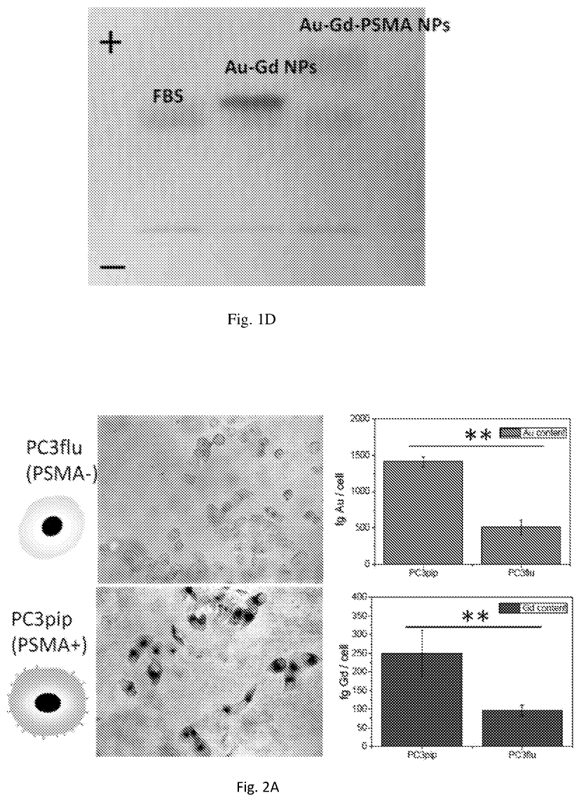

The nanoparticle agents described herein include a gold nanoparticle, at least one thiol modified gadolinium 3+(Gd(III)) macrocycle complex coupled to the gold nanoparticle, and at least one prostate specific membrane antigen (PSMA) ligand coupled to the gold nanoparticle for targeting the composition to a PSMA expressing cancer cell. The PSMA ligand and the thiol modified Gd(III) complex are coupled to the gold nanoparticle via one or more thiol (SH) groups. In an exemplary embodiment, the thiol modified gadolinium macrocycle complex and a PSMA ligand are each individually covalently bound to the surface of the gold nanoparticle using simple Au-thiol conjugation via thiol (SH) groups (see FIG. 1).

It was found that at least one amine functionalized and thiol modified Gd(III) macrocycle complex and at least one PSMA ligand can be directly coupled gold nanoparticles. The presence of the PSMA ligand allows for the nanoparticle agent to specifically bind to and/or complex with PSMA expressing cancer cells to target the nanoparticle contrast agents to the cancer cells as well as cancer cell metastasis, migrations, dispersals, and/or invasions in a subject.

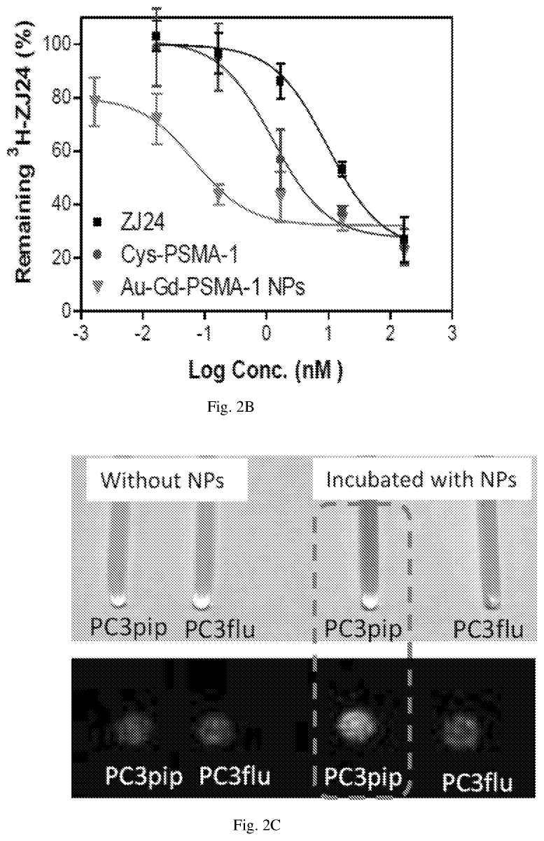

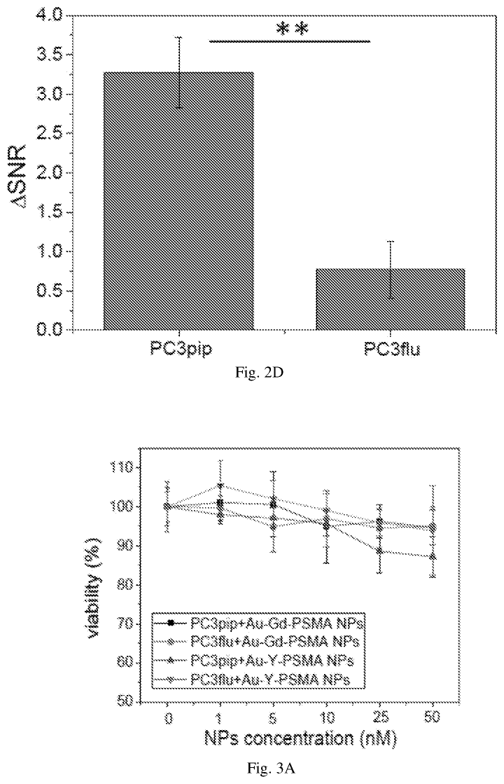

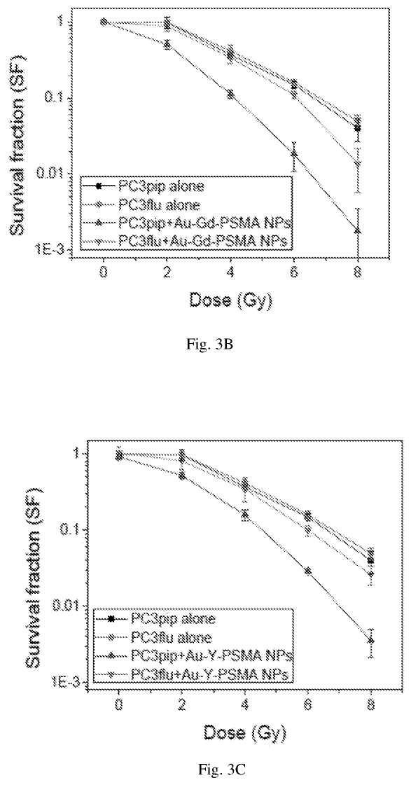

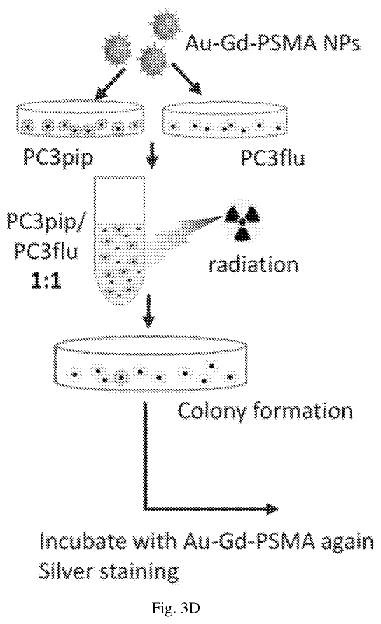

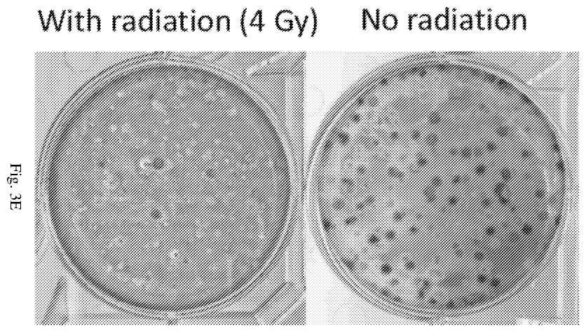

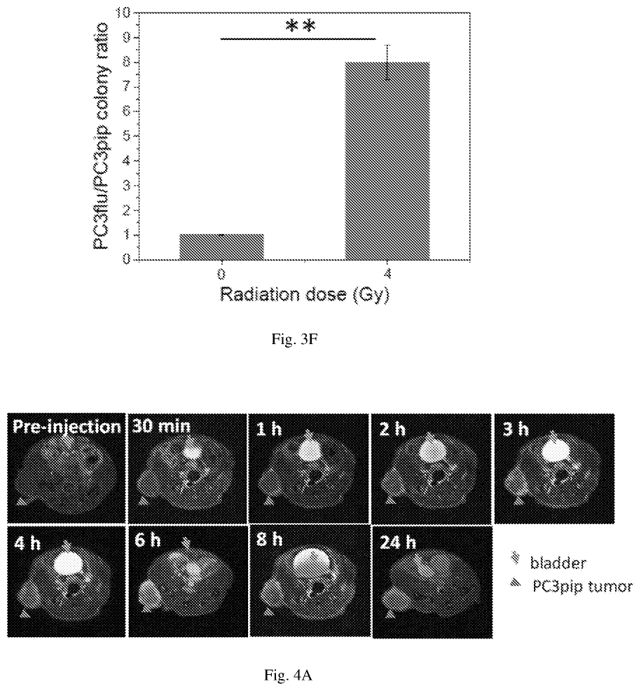

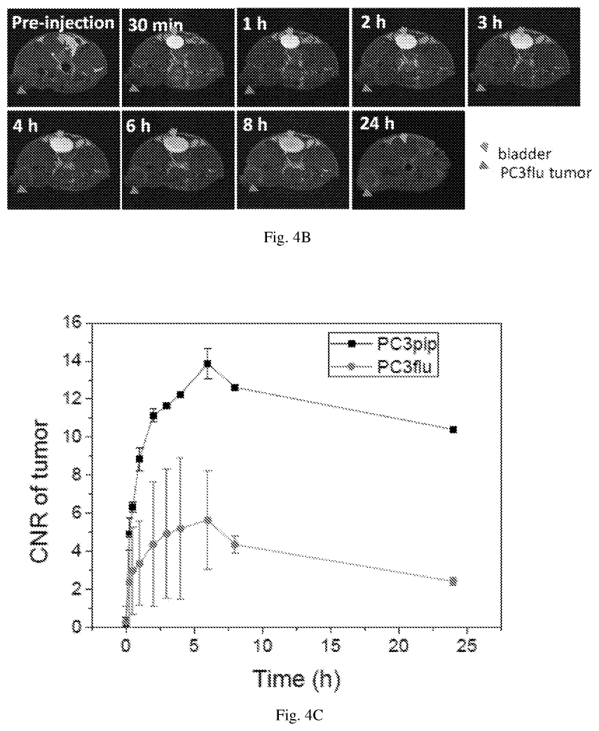

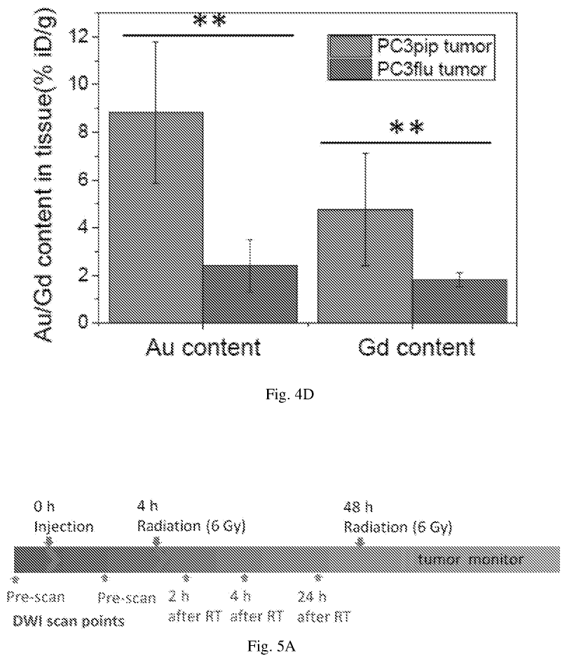

It was also found that gold nanoparticles with coupled PSMA ligands and thiol modified Gd(III) macrocycle complexes exhibit increased stability (see FIG. 2) and high relaxivity (see FIG. 3). The nanoparticle agents were also shown to selectively target and accumulate at PSMA expressing cancer tissue (see FIG. 4) where the nanoparticle agents can provide a synergistic sensitizing effect compared to gold particles or Gd macrocycle complexes administered alone (see FIG. 10).

Gold nanoparticle size can affect their biocompatibility, elimination rate, and will increase the relaxivity when Gd(III) complexes are conjugated to the surface. For example, smaller gold nanoparticles (e.g., having less than 6 nm core diameter) allow for favorable renal clearance and enhanced X-ray therapy in tumors that express the PSMA biomarker when administered to a subject. Therefore, in some embodiments, the gold nanoparticle of the nanoparticle agent is less than about 6 nm in core diameter. In a particular embodiment, the gold nanoparticle is about 2 nm to about 5 nm in core diameter. Particle size can be measured using dynamic light scattering (DLS) and TEM.

A thiol modified Gd(III) macrocycle complex can be modified using a chemical linker to add a terminal thiol group that allows for the complexes to be coupled to the gold nanoparticle via Au-thiol conjugation. In some embodiments, the Gd(III) macrocycle complex can include an amine functionalized to the chemical linker, which allows for conjugation of the Gd(III) macrocycle complex to a thiol. For example, the at least one thiol modified Gd(III) macrocycle complex can be amine functionalized to allow for conjugation of the Gd(III) macrocycle complex to a lipoic acid thiol. In particular embodiments, the at least one thiol modified Gd(III) macrocycle complex coupled to a gold nanoparticle can include an amine functionalized and 1,2 dithiolane modified Gd(III) macrocycle complex.

The thiol modified Gd(III) macrocycle complex coupled to the gold nanoparticle can include a chelating compound. The chelating group can reduce the longitudinal relaxation time of nearby water protons, which are aided by the high magnetic moment and symmetrical S state of the Gd(III) ion. A number of chelating compounds have been developed to increase the coordinated water molecules for lanthanide ions such as gadolinium, which can almost double the relaxivity rate. Examples of effective gadolinium chelating molecules include 1,4,7,10-tetraazacyclododecane-1,4,7,10-tetraacetic acid (DOTA), diethylenetriaminopentacetate (DTPA), 1,4,7,10-tetraazacyclododecane-1,4,7-triasacetic acid (DO3A), 6-amino-6-methylperhydro-1,4-diazepinetetraacetic acid (AAZTA), and 4-carboxyamido-3,2-hydroxypyridinone (HOPA). See Gugliotta et al., Org. Biomol. Chem., 8, 4569 (2010), the disclosure of which is incorporated herein by reference. In certain embodiments, the gadolinium chelating molecule for use in a thiol modified Gd(III) macrocycle complex described herein includes DOTA.

In some embodiments, the thiol modified Gd(III) macrocycle complex is an amine functionalized and 1,2 dithiolane modified Gd(III) macrocycle complex having the formula:

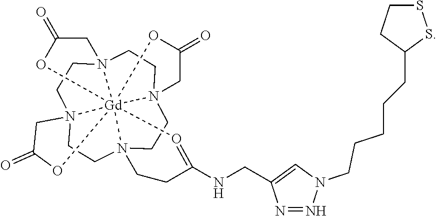

The PSMA ligand coupled to the gold nanoparticle can include a peptide-based PSMA ligand, (e.g., PSMA-1(Cys)), that has been thiol modified (e.g. using a cysteine residue) at the C-terminal end of the molecule to allow for coupling to a gold nanoparticle. It has been shown that PSMA targeted gold nanoparticles can increase uptake of the Au—Gd(III) nanoparticles in PSMA expressing cells while also improving cell killing compared to agents administered alone. In addition, PSMA targeted Au—Gd(III) nanoparticles described herein can decrease off-target toxicity of the agent administered (e.g., systemically) to a subject.

In some embodiments, the PSMA ligand coupled to the gold nanoparticle can have the general formula (I):

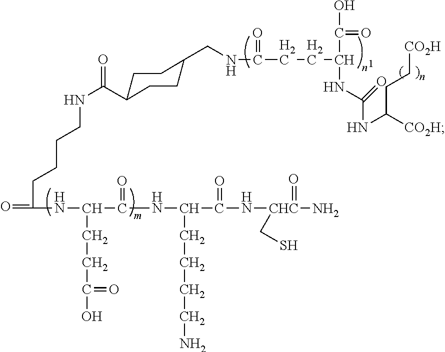

wherein m, n and n¹ are each independently 1, 2, 3, or 4.

In certain embodiments, the PSMA ligand coupled to a gold nanoparticle as described herein can have the formula (II):

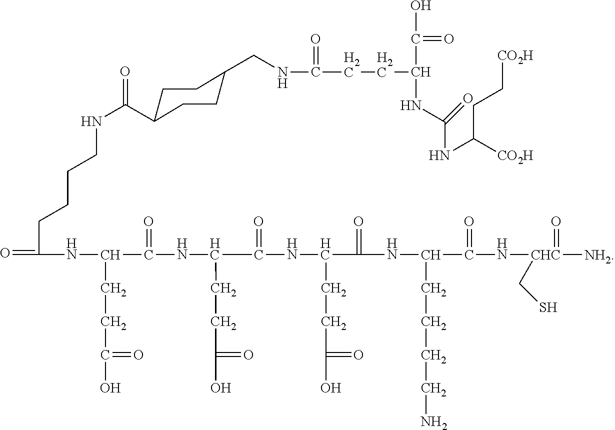

In some embodiments, gold nanoparticles with a core diameter size of less than 6 nm can be synthesized in a two-phase toluene-H₂O system, where PSMA ligand and Gd(III) macrocyclic complex are conjugated to the gold nanoparticle surface via ligand exchange resulting in a nanoparticle having an average hydrodiameter of about 7.8 nm. For example, gold nanoparticles having a core size of about 2 nm to about 5 nm can be synthesized using a modified Brust-Schriffin method. Next, a 1000-fold excess of Gd(III) complex can be added to a gold nanoparticle (Au—NP) suspension. After about one day of reaction, the solvent is removed and the nanoparticles are purified by centrifugation. The loading efficiency of the Gd(III) macrocycle complex onto the Au-NPs can be determined by collecting free unconjugated Gd(III) macrocycle complex for ICP-MS (inductive coupled plasma-mass spectrometry) measurement. After purification, the Au—Gd NPs are resuspended in H₂O, to which 40-fold excess of Cys-PSMA ligand is added. After incubation for another day, solvent can be removed and final PSMA-Au—Gd NPs can be purified by centrifugation. The number of Cys-PSMA on the particles can be quantified indirectly by measuring free Cys-PSMA ligand (i.e., PSMA-1) using HPLC. In addition, transmission electron microscopy (TEM), dynamic light scattering (DLS), UV-Vis spectroscopy, gel electrophoreses and ICP-MS can be used to characterize the size of the PSMA-Au—Gd NPs generated and to determine the effective final concentration of Gd(III) macrocycle complex on the surface of the PSMA targeted gold nanoparticles.

In an exemplary embodiment, the PSMA targeted gold nanoparticles generated can have a T1 relaxivity (r₁) of about 20.6 mM⁻¹ s⁻¹ at 37° C. (1.41 T) with a total surface loading of 230±10 Gd(III) complexes per particle. Nanoparticle agent relaxivities can be measured by a NMR minispec using well known methods.

It was found that PSMA-targeted gold nanoparticles can significantly improve cell uptake in PSMA positive cells thus enhancing the MRI contrast. When the PSMA targeted Au—Gd nanoparticle agents described herein are used as molecular probes, the nanoparticle agents can precisely localize and clearly demarcate the cancer cells in tissue sections and tumor “edge” samples, suggesting that the nanoparticle agents can be used as diagnostic tools for molecular imaging of metastatic, dispersive, migrating, or invading cancers or the tumor margin.

The agents described herein can therefore be used in a method of detecting cancer cells and/or cancer cell metastasis, migration, dispersal, and/or invasion as well as in methods of treating cancer in a subject in need thereof. The methods can include administering to a subject a diagnostically and/or therapeutically effective amount of a PSMA targeted Au—Gd NP agent described herein and detecting the nanoparticle agent selectively targeted to PSMA expressing cancer cells and/or cancer tissue.

Pathological studies indicate that PSMA is expressed by virtually all prostate cancers, and its expression is further increased in poorly differentiated, metastatic, and hormone-refractory carcinomas. Higher PSMA expression is also found in cancer cells from castration-resistant prostate cancer patients. Increased PSMA expression is reported to correlate with the risk of early prostate cancer recurrence after radical prostatectomy. In addition to being overexpressed in prostate cancer (PCa), PSMA is also expressed in the neovasculature of neoplasms including but not limited to conventional (clear cell) renal carcinoma, transitional cell carcinoma of the urinary bladder, testicular embryonal carcinoma, colonic adenocarcinoma, neuroendocrine carcinoma, gliobastoma multiforme, malignant melanoma, pancreatic ductal carcinoma, non-small cell lung carcinoma, soft tissue carcinoma, breast carcinoma, and prostatic adenocarcinoma.

In some embodiments, the PSMA targeted Au—Gd nanoparticle agents described herein, can selectively target and recognize PSMA-expressing tumors, cancer cells, and/or cancer neovasculature in vivo and be used to deliver Au—Gd NPs to the PSMA-expressing tumors, cancer cells, and/or cancer neovasculature for use as high-affinity radiosensitizers to detect and/or treat the PSMA-expressing tumors, cancer cells, and/or cancer neovasculature in a subject.

The nanoparticle agents can be administered systemically to a subject and selectively target PSMA-expressing cancer cells. In some embodiments, the nanoparticle agent after systemic administration can define PSMA-expressing cancer cell location, distribution, metastases, dispersions, migrations, and/or invasion as well as tumor cell margins in the subject. In other embodiments, the nanoparticle agent after systemic administration can be used to inhibit and/or reduce cancer cell survival, proliferation, and migration.

In some embodiments, the PSMA expressing cancer that is detected and/or treated is prostate cancer. In other embodiments, the cancer that is detected and/or treated can include malignant neoplasms, such a conventional (clear cell) renal carcinoma, transitional cell carcinoma of the urinary bladder, testicular embryonal carcinoma, colonic adenocarcinoma, neuroendocrine carcinoma, gliobastoma multiforme, malignant melanoma, pancreatic ductal carcinoma, non-small cell lung carcinoma, soft tissue carcinoma, breast carcinoma, and prostatic adenocarcinoma.

In some embodiments, the PSMA targeted Au—Gd nanoparticles described herein may be used in conjunction with non-invasive imaging (e.g., neuroimaging) techniques for in vivo imaging of the nanoparticle agents, such as magnetic resonance spectroscopy (MRS) or imaging (MRI), or gamma imaging, such as positron emission tomography (PET) or single-photon emission computed tomography (SPECT). The term “in vivo imaging” refers to any method, which permits the detection of a nanoparticle agent, as described above.

In certain embodiments, the nanoparticle agent is detected in a subject by magnetic resonance imaging (MRI). MRI relies upon changes in magnetic dipoles to perform detailed anatomic imaging and functional studies. MRI can employ dynamic quantitative T₁ mapping as an imaging method to measure the longitudinal relaxation time (i.e., the T₁ relaxation time) of protons in a magnetic field after excitation by a radiofrequency pulse. T₁ relaxation times can in turn be used to calculate the concentration of a nanoparticle agent serving as a molecular probe in a region of interest. The macrocycle-structured gadolinium(III)chelates of the nanoparticle agent are positive contrast agents (appearing predominantly bright on MRI) characterized as small molecular weight organic compounds that chelate or contain an active element having unpaired outer shell electron spins.

The nanoparticle agents described herein can be administered to the subject by, for example, systemic, topical, and/or parenteral methods of administration. These methods include, e.g., injection, infusion, deposition, implantation, or topical administration, or any other method of administration where access to the cells and/or tissue by the nanoparticle agent is desired. In one example, administration of the nanoparticle agent can be by intravenous injection of the nanoparticle agent in the subject. Single or multiple administrations of the nanoparticle agent can be given. “Administered”, as used herein, means provision or delivery of nanoparticle agent in an amount(s) and for a period of time(s) effective to label cancer cells in the subject.

Nanoparticle agents described herein can be administered to a subject in a diagnostically effective amount (e.g., a detectable quantity) of a pharmaceutical composition containing a nanoparticle agent or a pharmaceutically acceptable water-soluble salt thereof, to a subject. A “detectable quantity” means that the amount of the detectable compound that is administered is sufficient to enable detection of binding and/or uptake of the compound to the cancer cells. An “imaging effective quantity” means that the amount of the detectable compound that is administered is sufficient to enable imaging of binding and/or uptake of the compound to the cancer cells.

Formulation of the nanoparticle agent to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically, such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

The PSMA targeted Au—Gd nanoparticles administered to a subject can be used in a method to detect and/or determine the presence, location, and/or distribution of cancer cells expressing PSMA in an organ or body area of a patient, e.g., at least one region of interest (ROI) of the subject. The ROI can include a particular area or portion of the subject and, in some instances, two or more areas or portions throughout the entire subject. The ROI can include regions to be imaged for both diagnostic and therapeutic purposes. The ROI is typically internal; however, it will be appreciated that the ROI may additionally or alternatively be external.

The presence, location, and/or distribution of the nanoparticle agent in the animal's tissue, e.g., prostate tumor tissue, can be visualized (e.g., with an in vivo imaging modality described above). “Distribution” as used herein is the spatial property of being scattered about over an area or volume. In this case, “the distribution of cancer cells” is the spatial property of cancer cells being scattered about over an area or volume included in the animal's tissue, e.g., tumor tissue. The distribution of the molecular probe may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject. It will be appreciated that the imaging modality may be used to generate a baseline image prior to administration of the nanoparticle agent composition. In this case, the baseline and post-administration images can be compared to ascertain the presence, absence, and/or extent of a particular disease or condition.

In one aspect, the nanoparticle agent may be administered to a subject to assess the distribution of cancer cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of prostate on the tumor margin that are outside the tumor edge that appear to be grossly normal but are infiltrated by dispersing tumor cells upon histological examination. For example, in prostate cancer (prostate tumor) surgery, the molecular probes can be given intravenously prior to pre-surgical stereotactic localization MRI. The nanoparticle agents can be imaged on MRI sequences as a contrast agent that localizes with the prostate cancer tumor tissue.

Nanoparticle agents described herein that selectively target PSMA expressing cancer cells can be used in intra-operative imaging (IOI) techniques to guide surgical resection and eliminate the “educated guess” of the location of the tumor margin by the surgeon. It is anticipated that nanoparticle agents that function as diagnostic molecular imaging agents have the potential to increase patient survival rates.

In some embodiments, to identify and facilitate removal of cancers cells, microscopic intra-operative imaging (IOI) techniques can be combined with systemically administered or locally administered nanoparticle agents described herein. The nanoparticle agents upon administration to the subject can target and detect and/or determine the presence, location, and/or distribution of cancer cells, i.e., cancer cells expressing PSMA, in an organ or body area of a patient. In one example, the molecular probe can be combined with IOI to identify malignant cells that have infiltrated and/or are beginning to infiltrate at a tumor margin. The method can be performed in real-time during surgery. An imaging modality can then be used to detect and subsequently gather image data. The imaging modality can include one or combination of known imaging techniques capable of visualizing the nanoparticle agents. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micro-machine).

In one example, PSMA targeted Au—Gd nanoparticle agents can be applied as needed during surgery to interactively guide a surgeon and/or surgical instrument to remaining abnormal cells. The nanoparticle agents may be applied locally in low concentration, making it unlikely that pharmacologically relevant concentrations are reached. In one example, excess material may be removed (e.g., washed off) after a period of time (e.g., incubation period).

Another embodiment described herein relates to a method of monitoring the efficacy of a cancer therapeutic or cancer therapy administered to a subject. The methods and nanoparticle agents described herein can be used to monitor and/or compare the invasion, migration, dispersal, and metastases of a cancer in a subject prior to administration of a cancer therapeutic or cancer therapy, during administration, or post therapeutic regimen.

A “cancer therapeutic” or “cancer therapy”, as used herein, can include any agent or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy. This can provide a direct clinical efficacy endpoint measure of a cancer therapeutic. Therefore, in another aspect, a method of monitoring the efficacy of a cancer therapeutic is provided. More specifically, embodiments of the application provide for a method of monitoring the efficacy of a cancer therapy.

The cancer therapeutics can include therapeutic agents effective for the treatment of PSMA positive cancers. The cancer therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA.

The method of monitoring the efficacy of a cancer therapeutic can include the steps of administering in vivo to the animal a nanoparticle agent as described herein, then visualizing a distribution of the nanoparticle agents in the animal (e.g., with an in vivo imaging modality as described herein), and then correlating the distribution of the molecular probe with the efficacy of the cancer therapeutic. It is contemplated that the administering step can occur before, during, and after the course of a therapeutic regimen in order to determine the efficacy of a chosen therapeutic regimen. One way to assess the efficacy of the cancer therapeutic is to compare the distribution of a molecular probe pre and post cancer therapy.

In some embodiments, the PSMA targeted Au—Gd nanoparticle agents are detected in the subject to detect and/or provide the location and/or distribution of the cancer cells in the subject. The location and/or distribution of the cancer cells in the subject can then be compared to a control to determine the efficacy of the cancer therapeutic and/or cancer therapy. The control can be the location and/or distribution of the cancer cells in the subject prior to the administration of the cancer therapeutic and/or cancer therapy. The location and/or distribution of the cancer cells in the subject prior to the administration of the cancer therapeutic and/or cancer therapy can be determined by administering the nanoparticle agents to the subject and detecting the nanoparticle agents selectively targeted to PSMA expressing cancer cells in the subject prior to administration of the cancer therapeutic and/or cancer therapy.

In certain embodiments, the methods and nanoparticle agents described herein can be used to measure the efficacy of a therapeutic administered to a subject for treating a metastatic, invasive, or dispersed cancer. In this embodiment, the nanoparticle agents can be administered to the subject prior to, during, or post administration of the therapeutic regimen and the distribution of cancer cells can be imaged to determine the efficacy of the therapeutic regimen. In one example, the therapeutic regimen can include a surgical resection of the metastatic cancer and the nanoparticle agents can be used to define the distribution of the metastatic cancer pre-operative and post-operative to determine the efficacy of the surgical resection. Optionally, the methods and nanoparticle agents can be used in an intra-operative surgical procedure as described above, such as a surgical tumor resection, to more readily define and/or image the cancer cell mass or volume during the surgery.

It has been shown that combining a gold nanoparticle and gadolinium together in a nanoparticle agent described herein can synergistically enhance the radiosensitizing effect to potential ablation of cancer (e.g., prostate cancer) using a low radiation dose. In certain embodiments, the nanoparticle agent enables MRI-image guided radiation therapy to enhance radiation accuracy and to avoid collateral damage to normal tissues. Radiation can be administered to cancer cells or tissue using external beam radiotherapy. Radiation administered can include, but is not limited to gamma and X ray radiation.

In an exemplary embodiment, a radiation dose of 6 gy can be administered to the detected cancer cells or cancer tissue following the injection of the PSMA-targeted Au-GD NPs. In certain embodiments, the radiation dose is administered between about 2 to about 8 hours after injection of the nanoparticle agent. In a particular embodiment, the radiation dose can be administered about 4 hours after injection of the nanoparticle agent to the subject.

In an exemplary embodiment, MRI-image guided radiation therapy can be performed using an MRI LINAC device. An MRI-Linac device typically merges a high-strength MRI machine and a linear accelerator into a single device where the MRI machine provides real-time images of tumors as they are treated with radiation beams from the linear accelerator.

In other embodiments, an MRI machine and a linear accelerator can be used separately for MRI-image guided radiation therapy. High-field, diagnostic-quality MRI can provide visualization and detection of selectively targeted cancer cells or tumor tissue and the surrounding tissues and allows evaluation of response to treatments. Linear accelerators can be used to deliver high-precision radiation treatment to cancer cells and/or tumor tissue detected using a nanoparticle agent described herein. The integration of an MRI and LINAC into one machine for use in a method described herein allows for tracking and monitoring the movement of tumors during radiation delivery, and tracking radiation response in real-time, without any added radiation dose to the subject.

The nanoparticle agents described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The disclosed nanoparticle agents can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen. In specific embodiments, oral, parenteral, or systemic administration are preferred modes of administration for treatment.

The nanoparticle agents described herein can be administered alone as a monotherapy, or in conjunction with or in combination with one or more additional therapeutic agents. For example, the PSMA targeted Au—Gd nanoparticles described herein can be administered to the subject prior to, during, or post administration of an additional therapeutic agent and the distribution of metastatic cells can be targeted with the therapeutic agent. The agent can be administered to the animal as part of a pharmaceutical composition comprising the agent and a pharmaceutically acceptable carrier or excipient and, optionally, one or more additional therapeutic agents. The nanoparticle agents described herein and additional therapeutic agent can be components of separate pharmaceutical compositions, which can be mixed together prior to administration or administered separately. The nanoparticle agents described herein can, for example, be administered in a composition containing the additional therapeutic agent, and thereby, administered contemporaneously with the agent. Alternatively, the nanoparticle agents described herein can be administered contemporaneously, without mixing (e.g., by delivery of the agent on the intravenous line by which the therapeutic agent is also administered, or vice versa). In another embodiment, the nanoparticle agent described herein can be administered separately (e.g., not admixed), but within a short time frame (e.g., within 24 hours) of administration of the therapeutic agent.

The methods described herein contemplate single as well as multiple administrations, given either simultaneously or over an extended period of time. The nanoparticle agent described herein (or composition containing the agent) can be administered at regular intervals, depending on the nature and extent of the cancer's effects, and on an ongoing basis. Administration at a “regular interval,” as used herein, indicates that the therapeutically effective amount is administered periodically (as distinguished from a one-time dose). In one embodiment, the nanoparticle agent and/or an additional therapeutic agent is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. In some embodiments, the nanoparticle agent can be administered between, for example, once a day or once a week.

For example, the administration of the disclosed nanoparticle agent and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

The disclosed nanoparticle agent described herein and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions, the active ingredient will ordinarily be present in an amount of about 0.5-95% by weight based on the total weight of the composition.

The amount of disclosed nanoparticle agent described herein and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the nanoparticle agent described herein can also depend on the disease state or condition being treated along with the clinical factors and the route of administration of the compound.

For treating humans or animals, the amount of disclosed PSMA targeted Au—Gd nanoparticles and/or additional therapeutic agent administered (in milligrams of compound per kilograms of subject body weight) is generally from about 0.1 mg/kg to about 100 mg/kg, typically from about 1 mg/kg to about 50 mg/kg, or more typically from about 1 mg/kg to about 25 mg/kg. In a preferred embodiment, the effective amount of PSMA targeted Au—Gd(III) nanoparticles is about 1-10 mg/kg. In another preferred embodiment, the effective amount of agent or PSMA targeted Au—Gd(III) nanoparticles is about 1-5 mg/kg. The effective amount for a subject can be varied (e.g., increased or decreased) over time, depending on the needs of the subject. In an exemplary embodiment, the PSMA-targeted Au—Gd nanoparticles are administered via intravenous injection at a dose of 30 mg Au/kg. In other embodiments, the PSMA-targeted Au—Gd nanoparticles can be administered at a dose of 0.13 μmol NPs/kg body weight of a subject in need thereof.

The following examples are included to demonstrate preferred embodiments.

### EXAMPLE

In this Example, we synthesized PSMA-targeted AuNPs to selectively deliver Gd(III) contrast agents to prostate cancer and provide MR-guided radiation. By coupling a Gd(III) macrocyclic chelate directly to the surface of targeted AuNPs biomarker selectivity was achieved, the MR contrast sensitivity was significantly improved, and at the same time, both gold and gadolinium provided radiotherapeutic dose enhancement. Radiation after NP injection significantly inhibited tumor growth and diffusion-weighted imaging revealed tumor response to the Au—Gd(III)-PSMA NP-enhanced radiotherapy.

### Methods

### Materials

All materials were supplied by Sigma-Aldrich unless otherwise stated, and used without further purification.

### Synthesis and Characterization of the Gd(III) and Y(III) Complexes

The synthesis of the Gd(III) and Y(III) complexes proceeds via a convergent synthesis. The azido-labeled lipoic acid derivative (3-5(-azidopentyl)-1,2-dithiolane) and alkyne bearing DO3A derivative, are metallated with either Gd(III) or Y(III) coupled via copper(I)-catalyzed azide alkyne cycloaddition. Briefly, Cu(II)SO4, sodium ascorbate, and tris-hydroxypropyltriazolylamine were added to a 1:1:1 mixture of ethyl acetate, methanol, and water. To this solution was added 3-5(-azidopentyl)-1,2-dithiolane and the alkyne bearing DO3A derivative metallated with Gd(III) or Y(III). These complexes were purified by HPLC and the identity confirmed by analytical HPLC and high resolution ESI-TOF MS (Bruker AmaZon SL Ion Trap instrument).

Synthesis of the alkyne Gd(III) precursor proceeds from formation of N-propargyl acrylamide from propargylamine and acryloyl chloride. This acrylamide is then conjugated onto tert-butyl DO3A via an aza-Michael addition to yield the tert-butyl protected chelate. Following deprotection in 1:1 TFA and DCM, the chelate was metallated with GdCl3. The alkyne-modified contrast agent was isolated by reverse-phase HPLC. Synthesis of the lipoic azide proceeds by reduction of lipoic acid to the alcohol with BH3. The alcohol is converted to a tosylate group in situ, which is subsequently displaced by sodium azide. The final lipoic azide is purified via silica gel chromatography

### Synthesis of Au—Gd(III)-PSMA Nanoparticles

AuNPs with core size of 5 nm were synthesized using a modified Brust-Schiffrin method. After reaction, the DDA stabilized AuNPs were precipitated in pure ethanol and then re-suspended in chloroform. Concentration of AuNPs in chloroform was determined by UV-vis spectroscopy and a 1000 molar excess of Gd(III) complex was added to react with 1 equivalent of AuNP-DDA. The mixture was stirred over 24 h and then the solvent was evaporated at room temperature. The dried mixture was dissolved with PBS and un-conjugated Gd(III) complex was removed by extensive purification using centrifuge filters (MWCO=30 kDa, GE Healthcare). After purification, the Au— Gd(III) NPs were conjugated with Cys-PSMA-1 ligands with the same procedure with Cys-PSMA-1 added to Au—Gd(III) NPs at ratio of 40:1. After 24 h, un-conjugated peptides were removed by centrifugation with the 30 kDa filters. A similar procedure was used to generate Au— Y(III) NPs.

The hydrodynamic diameter of the Au—Gd(III)-PSMA NPs was characterized with a dynamic light scattering system (DynoPro NanoStar), and the AuNP core size was determined by transmission electron microscopy (FEI Tecnai F300 kV). Gel electrophoresis for PSMA-targeted Au—Gd(III)-PSMA NPs and untargeted Au—Gd(III) NPs was performed on 1% agarose gel and 1×TAE running buffer at 120 kV. Each chamber was loaded with 10 μL of 2 uM NPs, 5 μL of glycerol, and 5 μL of 4×TAE. All the NPs were pre-incubated with 10% fetal bovine serum (FBS) stained with coomassie brilliant blue (CBB) at 37° C. for 30 min. The stability of Au—Gd(III)-PSMA NPs in 10% FBS was monitor by UV-vis spectrometry over one month, and release of Gd(III) from NPs was monitored by inductively coupled plasma mass spectrometry (ICP-MS, Agilent technologies, 700 series). Au—Y(III) NPs were characterized similarly.

### Relaxivity and ICP-MS Measurements

Quantification of Gd(III) and Au was performed using ICP-MS. For sample preparation, NPs were digested in aqua regia (25% nitric acid and 75% hydrochloric acid) overnight. The coverage of Gd(III) per AuNPs were determined according to the Gd(III)/Au ratio from ICP-MS measurement. Au atom number (N) per NP was calculated using the equation: R=rs×N^{1/3} in which rs represents the Wigner-Seitz radius (rs=0.145 nm for Au) and R represents particle radius.

Relaxivity of Au—Gd(III) NPs and Au—Gd(III)-PSMA NPs were measured on a 1.4 T NMR minispec (Bruker). Serially diluted NPs solutions were heated to 37° C. and placed into the Bruker minispec mq60 NMR spectrometer (60 MHz) to measure the T1 relaxation time. An inversion recovery pulse sequence with 3 averages, 15 s repetition time and 7 data points was used for data collection. The inverse of the longitudinal relaxation time (1/T1, s1⁻¹) was plotted versus the Gd(III) concentration (mM), and the slope was defined as the relaxivity (Mm⁻¹ s⁻¹).

### Selective Cell Uptake

The selectivity of the Au—Gd(III)-PSMA NPs were determined by incubation with both the PSMA-positive PC3pip cells and PSMA-negative PC3flu cells. Cells were cultured in RPMI1640 medium with L-glutamine (2 mmol/L) and 10% FBS at 37° C. and 5% CO₂. To visualize the NP uptake cells were seeded in 8-well plates at 10⁴ cells per well and then co-incubated with 50 nM Au— Gd(III)-PMSA NPs for 24 h. Culture medium was then removed and cells were washed with PBS, fixed with 4% paraformaldehyde, stained with silver staining kits (sigma), and imaged with a Leica DM4000B fluorescence microscope (Leica Microsystem Inc.).

The amount of Au and Gd(III) uptake by cells was determined by ICP-MS. Both PC3pip and PC3flu cells were seeded in 6-well plates at 105 cells per well and incubated for 24 h. Then cells were trypsinized, washed with PBS, and counted before digesting with aqua regia overnight. The digested cell suspensions were then diluted with DI water and measured with ICP-MS.

### Binding Affinity of Au—Gd(III)-PSMA NPs

LNcap cells were used to test the binding affinity of NPs. Specifically, LNcap cells were cultured in RPMI1640 medium, harvested, washed with 0.5 mM cold Tris buffer, and divided into 1.5 mL Eppendorf tubes at 5×10⁵ cells per tube. The cells were then incubated with the ZJ24, free Cys-PSMA-1 ligands, and Au—Gd(III)-PSMA NPs at different concentrations in the presence of N—[N—[(S)-1,3-dicarboxypropyl]carbamoyl]-S-[3H]-methyl-L-cysteine (12 nmol/L, ³H-ZJ24, GE Healthcare Life Sciences) in Tri buffer for 1 h at 37° C. All the tubes were then centrifuged and cells were washed three times with cold PBS. Finally, EcoLume cocktail (4 mL, MP biomedicals) was added to each tube, and radioactivity was counted with a scintillation counter. The concentration of ligands required to inhibit 50% of binding (IC50) was determined using GraphPad Prism 3.0.

### Cell Pellet MR Imaging

Both PC3pip and PC3flu cells were incubated with 50 nM Au—Gd(III)-PSMA NPs for 24 h and then harvested with trypsin, washed with PBS, and transferred to 5¾″ flame-sealed Pasteur pipets. The pipets were centrifuged again at 100×g at 4.0° C. for 5 min to spin down the cell pellets. PC3pip and PC3flu cells without any NPs were used as the control. The samples were imaged using a RF RES 300 1H 089/023 quadrature transmit receive 23 mm volume coil (Bruker BioSpin, Billerica, MA, USA). For T1-weighted imaging, a spin echo sequence was used with the following parameters: TR=500 ms, TE=10 ms, flip angle=90°, NEX=3, FOV=20×20 mm 2, slice thickness=1 mm, and matrix size=256×256.

### In Vitro Cytotoxicity and Radiosensitization

Cytotoxicity of Au—Gd(III)-PSMA NPs and Au—Y(III)-PSMA NPs was evaluated with a CCK8 assay (Dojindo Molecular Technologies). Both PC3pip and PC3flu cells were cultured in 96-well plate and incubated with various concentration of NPs. Following a 24 h co-incubation, the medium was removed, and cells were washed with PBS. Fresh medium and CCK8 agent was added to each well. After 4 hours the 96-well plate was read at 450 nm (TECAN, infinite M200).

Radiosensitization of NPs was evaluated with a colony formation assay. Briefly, after incubating with 50 nM Au—Gd(III)-PSMA NPs or Au—Y(III)-PSMA NPs for 24 h, the PC3pip and PC3flu cells were washed with PBS to remove the non-internalized NPs, and then irradiated with X-ray (Cs-137 with energy of 0.6616 Mev) at doses of 0, 2, 4, 6 and 8 Gy. Next, the cells were harvested, counted, and seeded into 6-well plates. After incubating for 10 days, the cells were washed with PBS, fixed with 4% paraformaldehyde and stained with 0.4% crystal violet. The colony number was counted to calculate the surviving fraction. To determine the selective killing of PC3pip cells, an additional set of experiments was designed. PC3pip and PC3flu cells were individually administered Au—Gd(III)-PSMA NPs, incubated, and washed. Following washing the cells were combined together at a 1:1 ratio. The mixed cell suspension was irradiated with 4 Gy and reseeded into a 6-well plate. Following an additional incubation of 10 days, Au—Gd(III)-PSMA NPs were added again to label the cells expressing PSMA receptor (incubated for 24 h) and silver staining was carried out to verify the PC3pip colonies and PC3flu colonies.

### In Vivo Tumor Targeting and MR Imaging

All mice were handled and processed according to an approved protocol by Case Western Reserve University's IACUC (Animal Experimentation application 2015-003, approved Mar. 27, 2018-Mar. 27, 2021). Nude mice with flank tumors, PC3pip tumor or PC3flu tumor, were used to evaluate the active targeting of Au—Gd(III)-PSMA NPs and MR imaging. Mice (n=3) were injected (i.v.) with Au—Gd(III)-PSMA NPs at 60 μmol Gd(III)/kg body weight. Mice were imaged by MR on a Bruker Biospin 7 T magnet (Bruker Biospin, Billerica, MA, U.S.A.) before and 0.5 h, 1 h, 2 h, 3 h, 4 h, 6 h, 8 h and 24 h after injection using a spin echo sequence: TR=500 ms, TE=8.1 ms, flip angle=180°, NEX=3, FOV=20×20 mm², slice thickness=1 mm, and matrix size=256×256. The CNR of tumors was calculated as following: CNR=(tumor mean intensity−muscle mean intensity)/noise. Mice were euthanized after MR scanning and organs were discretized, weighed, lyophilized and digested with nitric acid to analyze both Au and Gd(III) content using ICP-MS.

### Radiation Therapy

When the PC3pip tumor reached a size about 100 mm³, mice were divided randomly into groups (n=5), which were injected with PBS, Au—Gd(III)-PSMA NPs or Au—Y(III)-PSMA NPs. Two different doses, 60 umol/kg and 30 umol/kg in terms of Gd(III) or Y(III), were injected (i.v.), and 4 hours after injection, the mice received 6 Gy of X-ray radiation focused onto the tumor area only. For another set of mice injected with either PBS or Au—Gd(III)-PSMA NPs (60 umol/kg), X-ray radiation (6 Gy) was given twice both at 4 h and 48 h after injection. All irradiated mice were monitored for tumor sizes and body weight every other day over 30 days.

### Diffusion-Weighted Imaging (DWI)

DWI images were acquired for each mouse. Briefly, mice were anesthetized in soflurane and placed in the prone orientation at isocenter in a Bruker Biospec 7.0T MRI scanner. Mice were maintained at 35+/−1 degree Celsius and 40-60 breaths throughout the imaging procedure. Following initial localizer axial DWI images were obtained for each animal's tumor using a DWI-EPI (echo planar imaging) acquisition (TR/TE=2000/27 ms. b=0 and 500 s/mm², FOV=17.6×16 mm, matrix=110×100, slice thickness=1 mm, 3 signal averages, and 4 EPI segment/TR).

All raw data was exported for offline analysis in Matlab (The Mathworks, Natick, MA). Apparent Diffusion Coefficient (ADC) maps were obtained for each imaging slice using established mon-exponential models. A region-of-interest analysis was then performed to measure the mean ADC value (in mm2/sec) for each tumor and imaging time point.

### Statistics

All the experiments were performed in triplicates unless stated otherwise. All numerical results are expressed as mean±SD. Descriptive statistics and significant differences between groups were analyzed using two-tailed student's t-tests, and the difference was considered significant if *p<0.05 and **p<0.01.

### Results

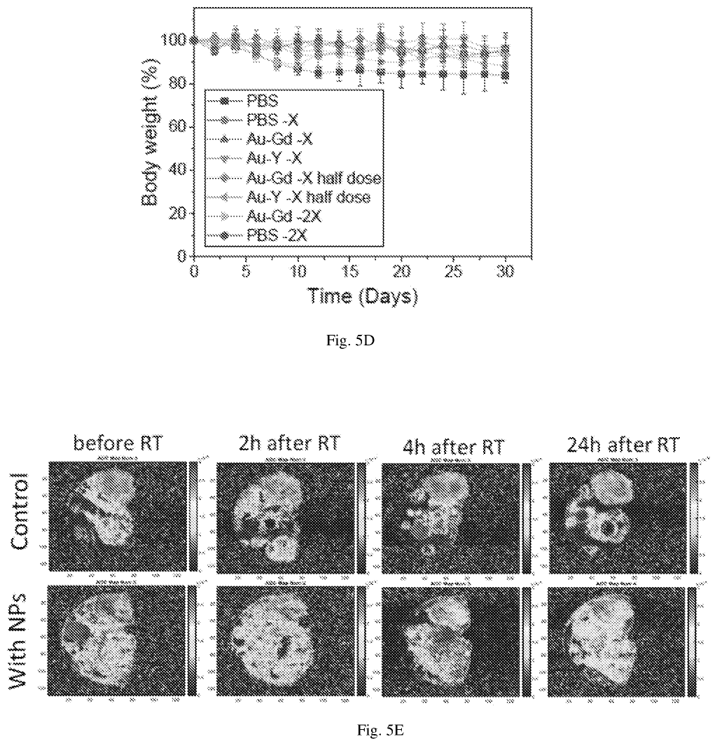

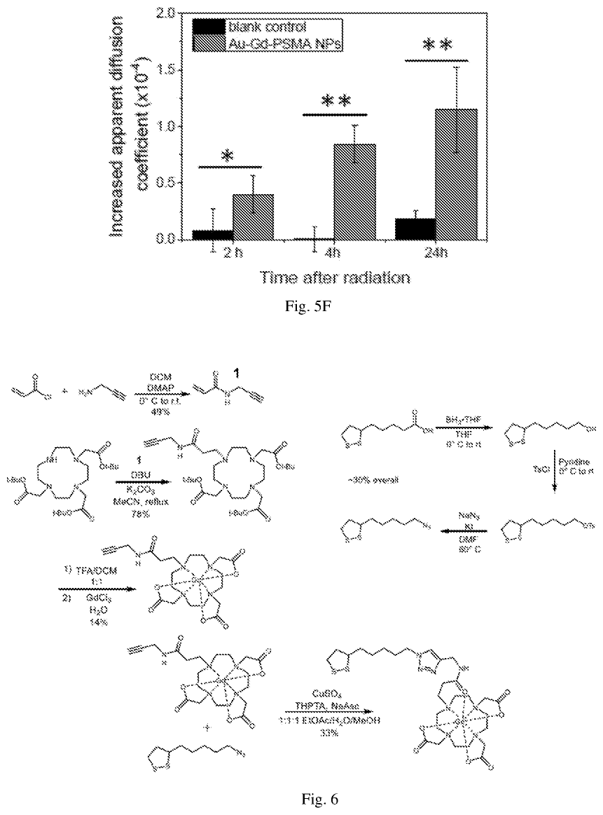

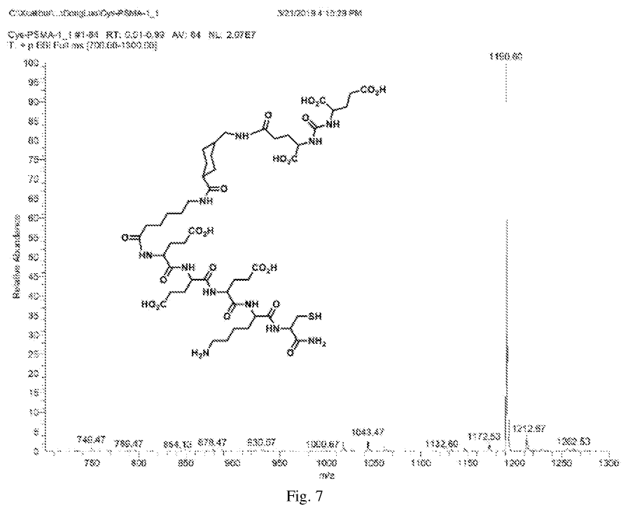

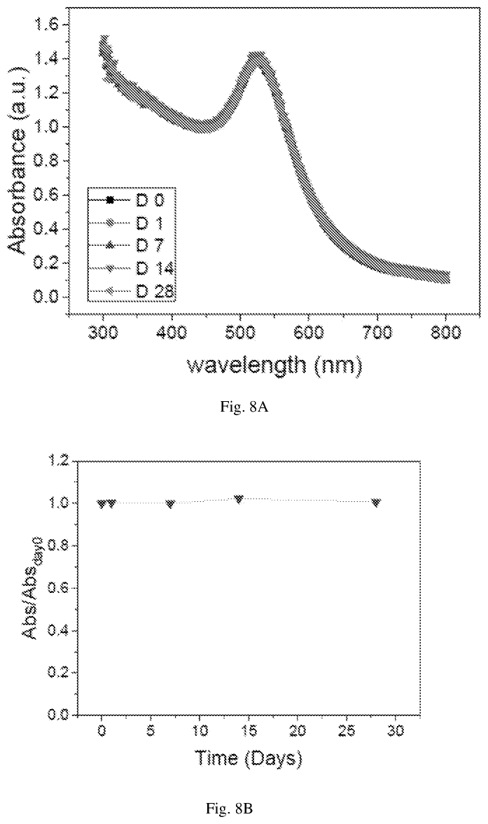

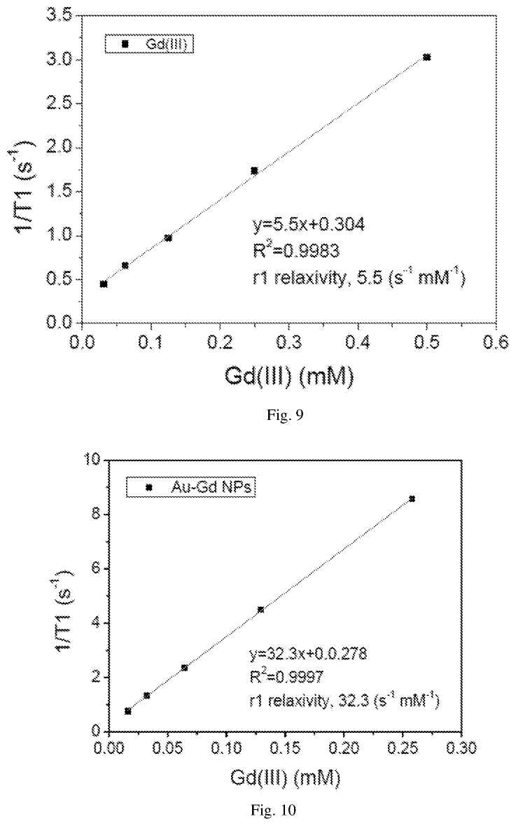

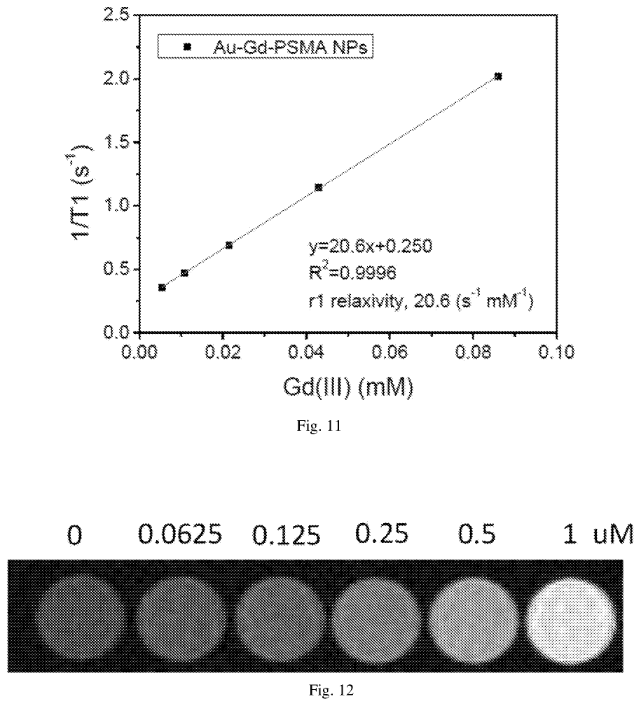

The Au—Gd(III)-PSMA NPs were synthesized by conjugating Gd(III) complex to the AuNP surface and active targeting of the Au—Gd(III)NPs was achieved by grafting Cys-PSMA-1 ligands as shown in FIG. 1A. The Gd(III) complex was synthesized as shown in FIG. 6. To verify the radiosensitizing effect of Gd(III), an analagous complex was synthesized with Yttrium, Y(III). The 1,2-dithiolane anchor with cyclic disulfide functionality has shown excellent surface binding affinity for gold, which binds the Gd(III) or Y(III) complex firmly to the AuNP surface. Moreover, the lipoic acid sequence of the Gd(III) complex improves the colloidal stability of our AuNPs allowing us to eliminate the need for PEGylation as a particle stabilizer and retaining the small the size of the Au—Gd(III)-PSMA NPs. TEM revealed that the Au—Gd(III)-PSMA NPs have a narrow core size distribution and an average diameter of 5 nm (FIG. 1B) and after conjugating Cys-PSMA-1 ligands, the hydrodynamic (HD) diameter was 7.8 nm (FIG. 1C). The stability of NPs in serum was monitored by gel electrophoresis, as described previously. After incubating with 10% FBS, a clear separation between the FBS band and the Au—Gd(III)-PSMA NPs band was observed, indicating limited irreversible serum adsorption to the particle surface (FIG. 1D). An increased mobility of the targeted AuNP towards the anode suggests the successful conjugation of Cys-PSMA-1 to AuNP, as PSMA-1 has a negative charge. The UV-vis absorbance of the NPs tested in PBS or serum over time confirmed long-term stability (FIG. 8).

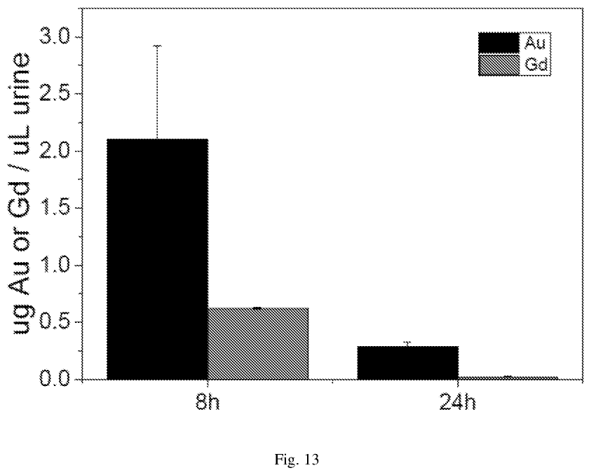

As shown in Table 1, the untargeted Au—Gd(III)NPs have a r1 of 32.3 mM⁻¹ s⁻¹ at 37° C. (1.4 T), with a total surface loading of 258±63 Gd(III) complexes per particle, and for the Au—Gd(III)-PSMA NPs r1=20.6 mM⁻¹ s⁻¹ at 37° C. (1.4 T), with a total surface loading of 230±10 Gd(III) complexes per particle. Both relaxivities are significantly higher than the ones for free Gd(III) complexes, which are only 5.5 mM⁻¹ s⁻¹ at 37° C. (FIGS. 13-1512, Table 2-4).

TABLE 1
Table 1 - Relaxivities of free Gd(III), Au—Gd(III)
NPs and Au—Gd(III)-PSMA NPs at 1.41

| | |
|---|---|
| | r₁ relaxivity (mM⁻¹s⁻¹) |
| Gd(III) loading | ionic particle |

| | per AuNP | Ionic | Particle |
|---|---|---|---|
| Gd(III) | NA | 5.5 | NA |
| Au—Gd(III) | 258 ± 63 | 32.3 | 8331 |
| Au—Gd(III)-PSMA | 230 ± 10 | 20.6 | 4745 |

“Ionic” r1 refers to the contribution of each individual Gd(III) complex to proton relaxation, whereas “particle” describes the product of each particle's Gd(III) payload.

TABLE 2
Measured values of T₁ and corresponding [Gd(III)]
measured by ICP-MS for Gd(III) complex

| sample | [Gd]/mM | T₁(ms) | T₁(s) | 1/T1 |
|---|---|---|---|---|
| 1 | 0.5 | 330 | 0.33 | 3.03 |
| 2 | 0.25 | 575 | 0.575 | 1.73 |
| 3 | 0.125 | 1030 | 1.03 | 0.97 |
| 4 | 0.0625 | 1510 | 1.51 | 0.66 |
| 5 | 0.03125 | 2232 | 2.232 | 0.45 |

TABLE 3
Measured values of T₁ and corresponding [Gd(III)]
measured by ICP-MS for Au—Gd NPs

| sample | [Gd]/mM | T₁ (ms) | T₁ (s) | 1/T₁ |
|---|---|---|---|---|
| 1 | 0.258 | 116.5 | 0.1165 | 8.58 |
| 2 | 0.129 | 221.9 | 0.2219 | 4.51 |
| 3 | 0.0645 | 424.1 | 0.4241 | 2.358 |
| 4 | 0.0323 | 753.5 | 0.7535 | 1.327 |
| 5 | 0.0161 | 1315.5 | 1.3155 | 0.760 |

TABLE 4
Measured values of T1 and corresponding [Gd(III)]
measured by ICP-MS for Au—Gd-PSMA NPs

| sample | [Gd]/mM | T₁ (ms) | T₁ (s) | 1/T₁ |
|---|---|---|---|---|
| 1 | 0.086 | 494.5 | 0.4945 | 2.022 |
| 2 | 0.043 | 874.5 | 0.8745 | 1.144 |
| 3 | 0.0215 | 1445 | 1.445 | 0.692 |
| 4 | 0.0108 | 2115 | 2.115 | 0.473 |
| 5 | 0.0054 | 2790 | 2.79 | 0.358 |

We tested the selectivity of Au—Gd(III)-PSMA NPs in vitro with both PSMA-positive PC3pip and PSMA-negative PC3flu cells. After co-incubating with Au—Gd(III)-PSMA NPs for 24 h, the PC3pip cells showed greater uptake than the PC3flu cells (FIG. 2A). PC3pip cells had almost a 3-fold higher Au uptake and 2.5-fold higher Gd(III) uptake than the PC3flu cells, indicating a PSMA receptor-mediated uptake of NPs. The binding affinity of Au—Gd(III)-PSMA NPs was determined by a competition binding assay. Au—Gd(III)-PSMA NPs, Cys-PSMA-1 alone, as well as the parent ligand ZJ24 were added to LNcap cell suspensions and incubated for 1 h at the presence of 3H-labelled ZJ24. The radioactivity retained by cells after extensive washes showed a remarkably lower IC50 of 0.07×10⁻⁹ M for the Au—Gd(III)-PSMA NPs compared to 1.26×10⁻⁹ M for the Cys-PSMA-1 and 9.79×10⁻⁹ M for the ZJ24. The significant improvement of binding affinity for the NPs is likely due to the multivalent binding effect, since multiple Cys-PSMA-1 ligands were covalently conjugated to the surface of each NP.

To verify that enhanced uptake of Au—Gd(III)-PSMA NPs would effectively translate to improved contrast in MR imaging, we harvested both the PC3pip and PC3flu cells after 24 h co-incubation with NPs and pelleted them in capillary tubes. PC3pip cell pellets incubated with Au—Gd(III)-PSMA NPs showed a visible pink color, originating from the NPs, which was absent for the PC3flu cell pellet (FIG. 2C). MR scanning at 7 T distinguished the PC3pip cells from the PC3flu cells and blank controls, showing an enhanced contrast in the T1-weighted image (FIG. 2C). From this image, the increased signal-to-noise ratio (ΔSNR) for PC3pip cells was calculated to be 3.3 which was significantly higher than 0.8 for the PC3flu cells (FIG. 2D). Clinical Gd(III)-based MRI contrast agents are usually not effectively internalized by cells. Conjugating Gd(III) contrast agents to targeted-NPs enhances the uptake into cells and likely will further improve the specificity for MR imaging.

Since both Au and Gd(III) have a high Z number and a notable mass energy absorption coefficient over soft tissues, we investigated the combination of these atoms on radiation enhancement. To demonstrate that, we synthesized AuNPs with Yttrium (Y) complex bound to the surface as a negative control (Y has little mass energy absorption): the chelated Gd(III) was replaced with Y (III) to ensure similar surface properties. First, we incubated PC3pip and PC3flu cells with various doses of Au—Gd(III)-PSMA NPs or Au—Y(III)-PSMA NPs for 24 h to evaluate the cytotoxicity. Neither of the NPs caused obvious toxicity to PC3pip or PC3flu cells with NP concentrations up to 50 Nm, FIG. 3A. Radiotherapy enhancement by NPs was then assessed by a colony formation assay. After incubating with 50 nM NPs for 24 h, both PC3pip and PC3flu cells were exposed to either 0, 2, 4, 6 or 8 Gy of X-ray radiation. Cells without NPs were irradiated with same doses. The sensitizing effect is dominated by the amount of high Z materials internalized by cells, though their intracellular distribution will also affect the radiotherapy outcome.

We hypothesized that an active NP uptake by PSMA targeting will sensitize cells to radiation, enhancing the radiation dose delivered to cells, and thus lead to more effective cell killing. As confirmed by the survival fraction curves (FIG. 3b), PC3pip cells incubated with Au—Gd(III)-PSMA NPs and irradiated showed a significantly lower survival rate compared to PC3flu cells or control cells at radiation doses above 2 Gy. Similar survival studies using Au—Y(III)-PSMA NPs (FIG. 3C) showed that the radiation enhancement of Au—Y(III)-PSMA NPs was likely due to Au only. Compared to PC3pip cells incubated with Au—Gd(III)-PSMA NPs, PC3pip cells incubated with Au—Y(III)-PSMA NPs had a slightly higher survival rate. The sensitization enhancement ratios (SER, the ratio of survival fractions without and with NPs at a survival level of 50%) for PC3pip cells incubated with Au—Gd(III)-PSMA NPs and Au—Y(III)-PSMA NPs were calculated to be 1.7 and 1.5, respectively, trending towards an increase in radiation sensitivity due to Gd(III) but not differing significantly. Presumably, this is because the amount of Gd(III) conjugated to the AuNP surface is much lower than the Au content in the NPs and thus much less Gd(III) is internalized into the cells.

To further demonstrate the selectivity of Au—Gd(III)-PSMA NPs to enhance killing of PSMA-expressing prostate cancer cells upon irradiation, we mixed equal amounts of NP-incubated PC3pip and PC3flu, irradiated the cell suspensions (4 Gy), and re-grew them in 6-well plates to form colonies. Before imaging the mixed colonies, we incubated them with NPs again and stained them with silver to distinguish the PC3pip and PC3flu colonies based on PSMA expression (FIG. 3D). For the cell mixtures without radiation, we measured equal amount of PC3pip (stained as black) and PC3flu (relatively transparent) colonies (FIG. 3E), with a ratio of 1 (FIG. 3F). In contrast, after irradiation, the PC3pip colony numbers were significantly reduced, with only a few black PC3pip colonies identifiable in the plate. There was little change in the number of PC3flu colonies. The PC3flu to PC3pip colony ratio increased to 8 after irradiation, suggesting that X-ray irradiation can selectively kill the PC3pip cells when Au—Gd(III)-PSMA NPs are internalized, even when they are very-well mixed with PC3flu cells. This selectivity feature is very important for clinical applications of radiation therapy since cancerous lesions are always adjacent to normal healthy tissues, underscoring that precisely targeted radiotherapy is urgently needed.

To evaluate the performance of Au—Gd(III)-PSMA NPs for prostate cancer imaging, animals were intravenously injected with Au—Gd(III)-PSMA NPs at 60 μmol Gd(III)/kg, which is about half of the standard dose for clinically used Gd(III)-DTPA and ⅕ of that for Gd(III)(HP-DO3A). Significantly increased contrast enhancement was observed for mice with PC3pip tumor for up to 24 h after NPs injection, peaking at approximately 6 h post-injection (FIG. 4A). There was limited contrast enhancement for the PC3flu tumor over 24 hours (FIG. 4B). There was also dramatic MR signal in the bladder, indicating renal clearance of Au—Gd(III)-PSMA NPs due to their small size. Quantitative analysis was done by subtracting the muscle signal from the tumor region of interest (ROI) and dividing by the standard deviation of the noise to generate the contrast-to-noise ratio (CNR) values. Au—Gd(III)-PSMA NPs produced a dramatic CNR increase of 13.9±0.8 for PC3pip tumors during the first 6 hours post-injection (FIG. 4C), while CNR for the PC3flu tumors did not increase significantly over time. The kinetics of CNR for PC3pip tumor has a similar trend to that of PSMA-targeted AuNPs accumulation in the tumors as revealed by CT scanning in our previous studies. The small size of the Au—Gd(III)-PSMA NPs facilitates rapid tumor extravasation, selective tumor binding, and sustained MR contrast, which enabled prostate cancer detection with significantly improved sensitivity.

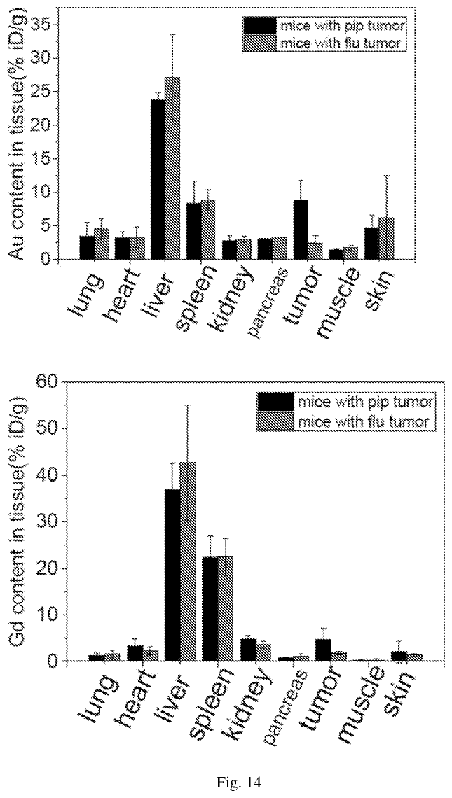

To further understand the performance observed in MR imaging, biodistribution of Au—Gd(III)-PSMA NPs was measured by ICP-MS at 24 h post-injection. Au and Gd(III) content in tumors and main organs were analyzed. As shown in FIG. 4D, there was significantly more Au and Gd(III) accumulation in PC3pip tumors than in PC3flu tumors, about 3.6-fold and 2.6-fold more for Au and Gd(III), respectively. This supports our hypothesis that active targeting of the prostate tumor via the PSMA receptor facilitates better NP accumulation compared to untargeted uptake, and thus provides enhanced MR contrast in targeted tumors. The minimal enhancement in the PC3flu tumors likely is due to EPR. Since significant MR signal was observed in bladder, we collected the urine at 8 h and 24 h post-injection and showed that large amount of Au and Gd(III) were detected in urine especially at 8 h (about 2.1 μg Au and 0.6 μg Gd(III) per μL urine, FIG. 13). The critical hydrodynamic diameter for NPs to get efficiently filtered by the glomerulus in the kidney is <6 nm, whereas hydrodynamic diameters>8 nm cannot primarily undergo renal clearance, and the renal excretion for these with hydrodynamic diameters in the range of 6-8 nm is dependent on their surface charge. This explains our presented observations for the currently studied Au—Gd(III)-PSMA NPs. While they can be excreted renally, many of them end up in the reticuloendothelial system (RES) as significant accumulation of Au—Gd(III)-PSMA NPs in the liver and spleen was observed (FIG. 14), suggesting additional clearance pathways via the RES and digestive systems.

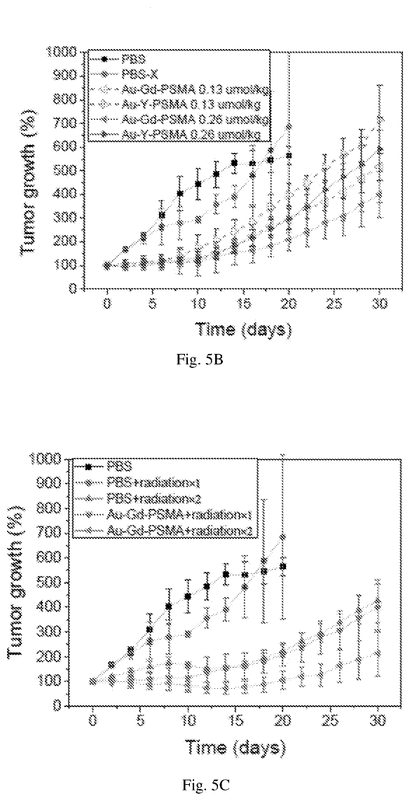

To investigate the potential use of the Au—Gd(III)-PSMA NPs for radiotherapy of prostate cancer, mice bearing a PC3pip tumor were injected with either Au—Gd(III)-PSMA NPs or Au—Y(III)-PSMA NPs, and radiation (6 Gy) was given once at 4 h or twice at both 4 h and 24 h post-injection (FIG. 5A). Mice injected with PBS and receiving the same treatment were used as controls. Tumor size and mouse body weight was then monitored for 30 days. In contrast to PBS control, both types of NPs resulted in obvious reduction in tumor growth (FIG. 5B) suggesting enhancement of X-ray irradiation. Radiation enhancement was measured with NP doses increasing from 0.13 μmol NPs/kg (dash lines) to 0.26 μmol NPs/kg (solid lines), and was dose-dependent for both types of NPs. Comparing the growth curves of tumors receiving the two types of NPs, Au—Gd(III)-PSMA NPs had significantly better tumor inhibition than Au—Y(III)-PSMA NPs, suggesting complementary radiosensitization by the combination of Au and Gd(III). However, giving only one irradiation at 4 h after NP injection did not completely inhibit the prostate tumor growth and resulted in tumors growing back rapidly after two weeks. We therefore tested multiple X-ray irradiations, which is often performed in clinical radiotherapy of prostate cancer.

At 48 h post NP injection we performed a second X-ray irradiation. In tumor-bearing mice injected with Au—Gd(III)-PSMA NPs, a second irradiation significantly reduced tumor growth, which only increased in size by 114% by day 30, compared to 300% for tumor-bearing mice injected with the same dose of NPs but receiving only one irradiation (FIG. 5C). Body weight for all the mice receiving radiation did not show significant changes, indicating that the NPs were safe to use for radiotherapy. For mice without irradiation, the body weight dropped to 84% (FIG. 5d) indicating progressing disease.

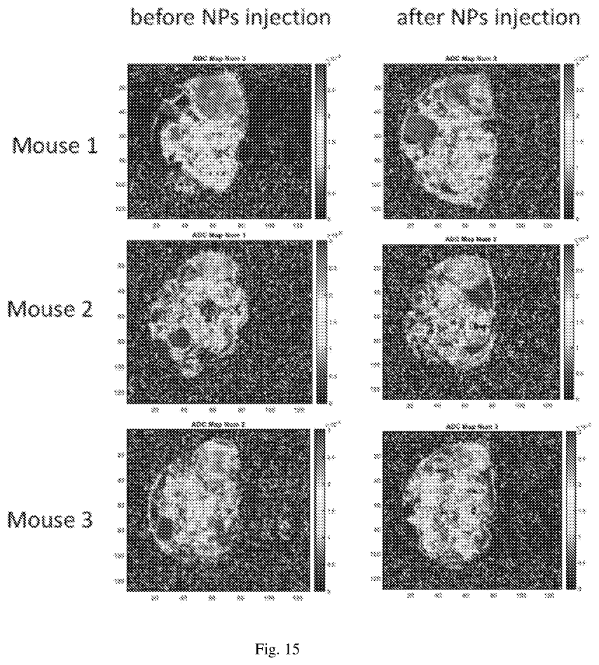

To further demonstrate the changes induced by radiation in tumors, we carried out a diffusion-weighted imaging (DWI) scan before and after irradiation, and the apparent diffusion coefficient (ADC) maps were acquired (FIG. 5E). ADC is a direct reflection of water proton mobility. Since increased tumor necrosis can promote water molecule diffusion in tumors and result in enhanced ADC values, we utilized DWI to further evaluate tumor treatment outcome. Alone the Au— Gd(III)-PSMA NP injection did not cause any changes to the ADC values (FIG. 15), but after irradiation the ADC in the tumors was significantly increased 24 hours after X-ray irradiation (FIG. 5E). In contrast, irradiation alone without any NP injection, did not cause any difference in ADC values. The changes of ADC value are plotted in FIG. 5F, show that with NP injection, irradiation increased the ADC significantly by 1.15×10⁻⁴ mm²/sec after 24 h, whereas for the blank control, it increased marginally by 0.18×10⁻⁴ mm²/sec. These DWI results suggest that the Au— Gd(III)-PSMA NP-enhanced radiotherapy for prostate cancer is based on destruction of the targeted cancer cells. This method could be used to monitor radiotherapy outcomes quantitatively and noninvasively during the radiotherapy.

In summary, we have described actively targeted Au—Gd(III)-PSMA NPs for prostate cancer MR imaging and radiotherapy. Both the Au and Gd(III) atoms can serve as radiosensitizers, and the conjugation of Gd(III) complexes onto AuNP surfaces improved MR sensitivity about 4-fold. The targeted Au—Gd(III)-PSMA NPs were prepared by conjugating Gd(III) complexes and a prostate-specific membrane antigen targeting ligand (Cys-PSMA-1) onto the AuNP surface. This surface modification increased the r1 relaxivity by a factor of four and also led to a higher binding affinity. The Au—Gd(III)-PSMA NPs have an excellent selectivity to PSMA expressing prostate cancer cells and thus enhanced the MR contrast of cells and radiosensitization in vitro. Systemically administered Au—Gd(III)-PSMA NPs showed good tumor accumulation, MR contrast, and significant in vivo radiation dose amplification. With high prostate cancer targeting specificity, MR contrast sensitivity and renal clearance, the Au—Gd(III)-PSMA NPs can inform future clinical MR-guided radiotherapy of prostate cancer. Ultimately, these particles may be used to lower the therapeutic X-ray dose, protecting normal surrounding tissue from radiation damage, while allowing cancer cell destruction. Specifically, radiotherapy is moving towards MR-LINAC for therapy of several different cancers including prostate and pancreatic cancer. The development of such a targeted MR imaging radio sensitizer may play a significant role in the development of MR-LINAC approaches. Significantly, PSMA receptor is overexpressed in the neovasculature of most solid human tumors making the application of PSMA-targeted NP developed here to have a much broader application for radiotherapy than just prostate cancer.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

Having described the invention, the following is claimed:

1. A nanoparticle agent for use in detecting, monitoring, and/or imaging cancer cells and/or cancer cell metastasis, migration, dispersal, and/or invasion, and/or for treating cancer in a subject, the nanoparticle agent comprising:
a gold nanoparticle;
at least one thiol modified Gd(III) macrocycle complex coupled to the gold nanoparticle; and
at least one prostate specific membrane antigen (PSMA) ligand coupled to the gold nanoparticle for targeting the composition to a PSMA expressing cancer cell,
wherein the PSMA ligand and the Gd(III) complex are each individually directly coupled to the gold nanoparticle via one or more thiol (SH) groups.

2. The nanoparticle agent of claim 1, the at least one thiol modified Gd(III) macrocycle complex comprising a lipoic acid modified and amine functionalized Gd(III) macrocycle complex.

3. The nanoparticle agent of claim 2, the lipoic acid comprising a 1,2 dithiolane.

4. The nanoparticle agent of claim 2, wherein the at least one thiol modified Gd(III) macrocycle complex has the formula:

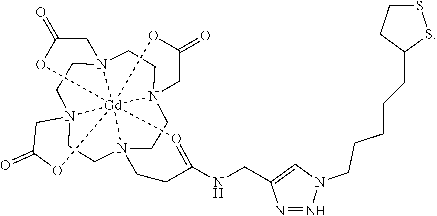

5. The nanoparticle agent of claim 1, wherein the PSMA ligand includes general formula (I): 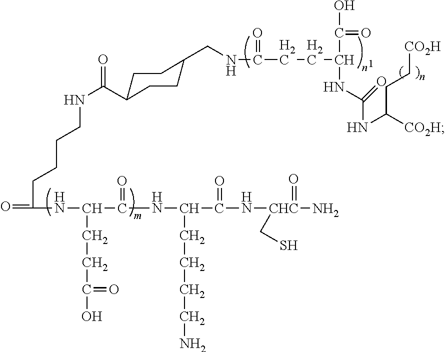
wherein m, n and n¹ are each independently 1, 2, 3, or 4.

6. The nanoparticle agent of claim 1, the PSMA ligand having the formula (II):

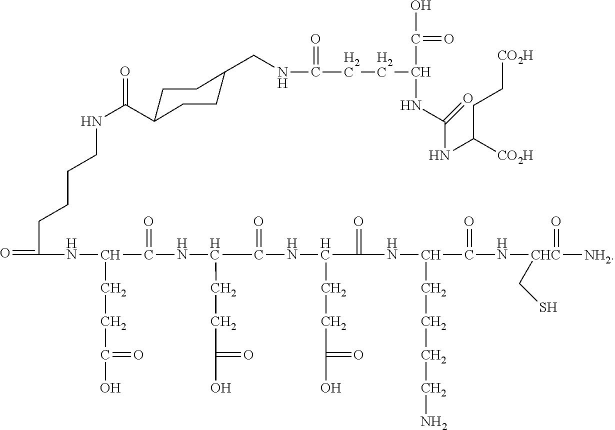

7. The nanoparticle agent of claim 1, wherein the gold nanoparticle is less than about 6 nm in core diameter.

8. The nanoparticle agent of claim 1, wherein the agent is detectable using magnetic resonance imaging (MRI).

9. The nanoparticle agent of claim 1, the cancer cell comprising a PSMA expressing cancer cell.

10. The agent of claim 9, the PSMA expressing cancer cell comprising at least one of a glioma, retinoblastoma, lung cancer, melanoma, breast cancer, ovarian cancer, endometrial cancer, and prostate cancer cell.

11. A method of detecting, monitoring, and/or imaging cancer cells and/or cancer cell metastasis, migration, dispersal, and/or invasion, in a subject, the agent comprising:
(a) administering to a subject with cancer a diagnostically effective amount of a nanoparticle agent, the nanoparticle agent comprising:
a gold nanoparticle;
at least one thiol modified Gd(III) macrocycle complex coupled to the gold nanoparticle; and
at least one prostate specific membrane antigen (PSMA) ligand coupled to the gold nanoparticle for targeting the nanoparticle agent to a PSMA expressing cancer cell, wherein the PSMA ligand and the Gd(III) complex are each individually coupled to the gold nanoparticle via one or more thiol (SH) groups; and
(b) detecting the nanoparticle agent selectively targeted to the cancer cells to determine the location and/or distribution of the cancer cells in the subject.

12. The method of claim 11, the at least one thiol modified Gd(III) macrocycle complex comprising a lipoic acid modified and amine functionalized Gd(III) macrocycle complex.

13. The method of claim 12, the lipoic acid comprising a 1,2 dithiolane.

14. The method of claim 11, wherein the at least one thiol modified Gd(III) complex has the formula:

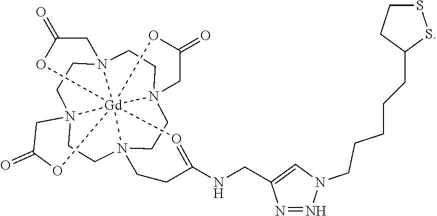

15. The method of claim 11, wherein the PSMA ligand includes general formula (I): 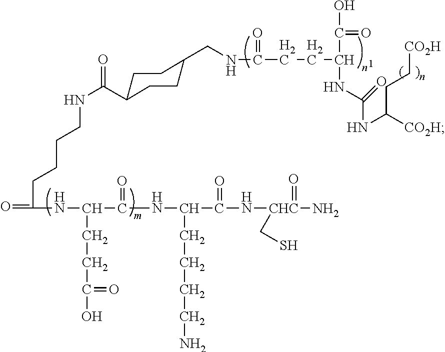
m, n and n¹ are each independently 1, 2, 3, or 4.

16. The method of claim 11, the PSMA ligand having the formula (II):

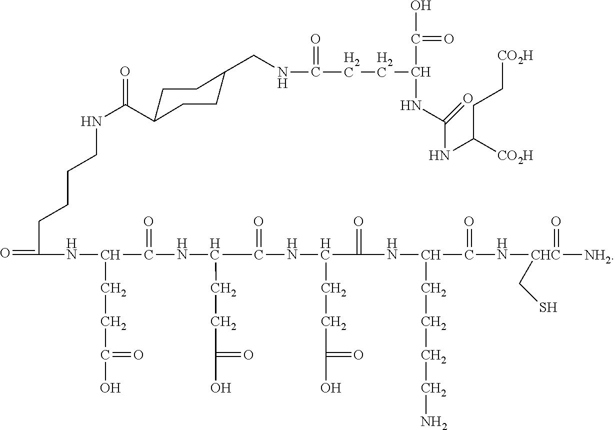

17. The method of claim 11, wherein the gold nanoparticle is less than about 6 nm in core diameter.

18. The method of claim 11, the cancer cell comprising a PSMA expressing cancer cell.

19. The method of claim 11, the PSMA expressing cancer cell comprising at least one of a glioma, retinoblastoma, lung cancer, melanoma, breast cancer, ovarian cancer, endometrial cancer, and prostate cancer cell.

20. The method of claim 11, the nanoparticle agent being detected by at least one of magnetic resonance imaging (MRI), positron emission tomography (PET) imaging, computer tomography (CT) imaging, gamma imaging, near infrared imaging, or fluorescent imaging.

21. The method of claim 11, wherein the nanoparticle agent is detected by MRI.

22. The method of claim 11, wherein the nanoparticle agent is administered systemically.

23. The method of claim 22, wherein the nanoparticle agent is administered by intravenous injection.

24. A method for treating cancer in a subject in need thereof comprising:
(a) administering to a subject with cancer a therapeutically effective amount of a nanoparticle agent, the nanoparticle agent comprising:
a gold nanoparticle;
at least one thiol modified Gd(III) macrocycle complex; and
at least one prostate specific membrane antigen (PSMA) ligand coupled to the gold nanoparticle for targeting the nanoparticle agent to a PSMA expressing cancer cells in the subject,
wherein the PSMA ligand and the Gd(III) complex are each individually coupled to the gold nanoparticle via one or more thiol (SH) groups;
(b) detecting nanoparticle agents selectively targeted to the cancer cells to determine the location and/or distribution of the cancer cells in the subject; and
(c) irradiating the cancer cells, thereby inducing the radiosensitizing effects of the nanoparticle agents.

25. The method of claim 24, the at least one thiol modified Gd(III) macrocycle complex comprising a lipoic acid modified and amine functionalized Gd(III) macrocycle complex.

26. The method of claim 25, the lipoic acid comprising a 1,2 dithiolane.

27. The method of claim 24, wherein the at least one thiol modified Gd(III) complex has the formula:

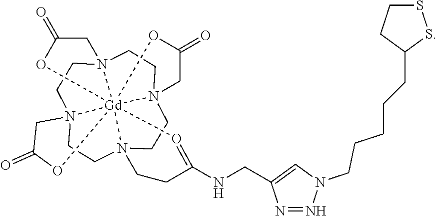

29. The method of claim 24, the nanoparticle agent comprising a prostate specific membrane antigen (PSMA) ligand having the general formula (I): 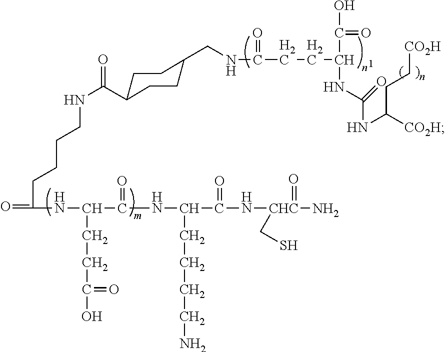
m, n and n¹ are each independently 1, 2, 3, or 4.

30. The method of claim 29, the PSMA ligand having the formula (II):

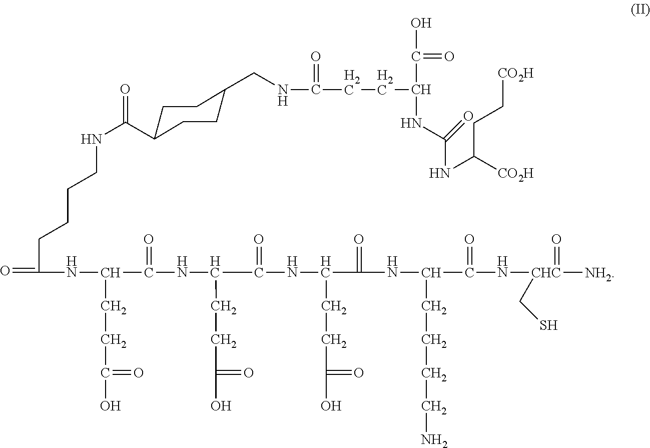

31. The method of claim 24, wherein the nanoparticle agent is administered systemically.

32. The method of claim 24, wherein the nanoparticle agent is administered by intravenous injection.

33. The method of claim 24, wherein the cancer is a PSMA expressing cancer.

34. The method of claim 33, wherein the PSMA expressing cancer is selected from glioma, retinoblastoma, lung cancer, melanoma, breast cancer, ovarian cancer, endometrial cancer, and prostate cancer.

35. The method of claim 34, wherein the PSMA expressing cancer is prostate cancer.

36. The method of claim 24, wherein the presence of the nanoparticle agent is detected in the subject by at least one of magnetic resonance imaging positron emission tomography (PET) imaging, computer tomography (CT) imaging, gamma imaging, near infrared imaging, or fluorescent imaging.

37. The method of claim 24 wherein the cancer is irradiated with gamma ray irradiation.

38. The method of claim 24, wherein the Gd(III) macrocycle complex coupled to the gold nanoparticle surface provides a synergistic effect on the radiosensitization of cancer cells.
